(19) United States
(12) Patent Application Publication
(10) Pub. No.: US 2014/0113297 A1
(43) Pub. Date: Apr. 24, 2014

(54) GENE EXPRESSION PREDICTORS OF CANCER PROGNOSIS

(71) Applicants: Joshi Alumkal, Portland, OR (US); Shannon K. McWeeney, Portland, OR (US)

(72) Inventors: Joshi Alumkal, Portland, OR (US); Shannon K. McWeeney, Portland, OR (US)

(73) Assignee: OREGON HEALTH & SCIENCE UNIVERSITY, Portland, OR (US)

(21) Appl. No.: 14/007,527

(22) PCT Filed: Mar. 23, 2012
(86) PCT No.: PCT/US2012/030309

§ 371 Date: Dec. 13, 2013

Related U.S. Application Data
(60) Provisional application No. 61/467,999, filed on Mar. 26, 2011.

(51) Int. Cl. C12Q 1/68

(52) U.S. Cl. CPC C12Q 1/6886
USPC 435/6.12

(57) ABSTRACT

Disclosed herein are methods of predicting the prognosis of a subject with prostate cancer. The methods include determining the expression level of a gene product of one or more of ZWILCH, DEPDC1, TPX2, CDCA3, HMGB2, MYC, CDC20, and/or KIF11. Expression of the gene product above a threshold level of expression indicates a poor prognosis such as a likelihood of relapse.

### PRIORITY CLAIM

This application claims the benefit of U.S. Patent Application No. 61/467,999 filed 26 Mar. 2011, which is hereby incorporated by reference in its entirety.

### ACKNOWLEDGEMENT OF GOVERNMENT SUPPORT

This invention was made with government support under grant numbers KL2 RR024141 and Pacific Northwest Prostate Cancer SPORE 2 P50 CA097186 awarded by the National Institutes of Health. The government has certain rights in the invention.

### FIELD

This disclosure relates to the field of cancer and particularly to methods for diagnosing and determining the prognosis of patients with a tumor.

### BACKGROUND

Cancer of the prostate is the most commonly diagnosed cancer in men and is the second most common cause of cancer death (Jemal et al, CA Cancer J Clin 59, 225-249 (2009) incorporated by reference herein.) If detected at an early stage, prostate cancer is potentially curable. However, a majority of cases are diagnosed at later stages when metastasis of the primary tumor has already occurred (Wang et al, Meth Cancer Res 19, 179 (1982) incorporated by reference herein.)

Even early diagnosis is problematic because not all individuals who test positive in these screens develop cancer. Furthermore, many prostate cancer patients are destined to develop fatal, metastatic castration-resistant prostate cancers (CRPC) that progress despite androgen deprivation therapy (ADT). It is now known that androgens and androgen-dependent signaling pathways modulated by the androgen receptor (AR) persist in some CRPC cells despite ADT (Mohler et al, Clin Cancer Res 25 10, 440-448 (2004) and Mostaghel et al, Cancer Res 67, 5033-5041 (2007) both of which are incorporated by reference herein.) However, these pathways may not account for progression of all CRPC cells. While newer and more potent forms of ADT benefit some patients with CRPC, the effect is not sustained, and in some patients there is no benefit at all (Scher et al, Lancet 375, 1437-1446 (2010).

### SUMMARY

Effective markers that predict prostate cancer outcome are unavailable. Disclosed herein are methods of determining prognosis of a subject with a tumor (such as a prostate tumor). In some embodiments, the methods include detecting expression of a gene selected from the group consisting of TPX2, microtubule associated homolog (TPX2); kinesin family member 11 (KIF11); Zwilch, kinetochore associated, homolog (ZWILCH); v-myc myelocytomatosis viral oncogene homolog (MYC); DEP domain containing 1 (DEPDC1); cell division cycle associated 3 (CDCA3); high-mobility group box 2 (HMGB2); cell division cycle 20 homolog (CDC20); and combinations of any two or more thereof, in a sample from the subject; and comparing expression of the gene(s) in the sample to a control sample, wherein an increase in expression of at least one of the gene(s) relative to the control indicates that the subject has a poor prognosis. In an example, the methods include detecting expression of at least two (such as at least 3, 4, 5, 6, 7, or all) of TPX2, KIF11, ZWILCH, MYC, DEPDC1, CDCA3, HMGB2, and CDC20 in a sample from the subject. In other examples, the methods include detecting expression of at least one gene listed in Table 1 and comparing expression of the gene in the sample to a control sample, wherein an increase in expression of the gene relative to the control indicates that the subject has a poor prognosis.

In some embodiments, a poor prognosis includes a decreased probability of survival, such as decreased overall survival, decreased metastasis-free survival, or decreased relapse-free survival. In another embodiment, a poor prognosis includes resistance or likelihood of developing resistance to a therapy (such as hormone therapies like ADT.) Alterations in gene expression can be measured using methods known in the art, and this disclosure is not limited to particular methods. For example, expression can be measured at the nucleic acid level (such as by quantitative reverse transcription polymerase chain reaction or micro array analysis) or at the protein level (such as by Western blot or other immunoassay analysis).

Also disclosed are arrays for determining prognosis of a subject with cancer, such as prostate cancer. In some embodiments, the array is a solid support including a plurality of agents (such as probes and/or antibodies) that can specifically detect one or more (such as 1, 2, 3, 4, 5, 6, 7, or all) of TPX2, KIF11, ZWILCH, MYC, DEPDC1, CDCA3, HMGB2, and CDC20 nucleic acids or proteins. In other embodiments, the array is a solid support including a plurality of agents (such as probes and/or antibodies) that can specifically detect one or more of the genes in Table 1. Arrays can also include other molecules, such as positive (including housekeeping genes) and negative controls as well as other cancer prognosis related molecule. The foregoing and other features of the disclosure will become more apparent from the following detailed description, which proceeds with reference to the accompanying figures.

### BRIEF DESCRIPTION OF THE DRAWINGS

FIG. 1 is a heatmap for probesets with an androgen receptor (AR) binding site within 50 kb of the annotated transcriptional start site in LNCaP and Abl cells. Expression data was robust multi-array average processed before fold changes were computed versus the controls. The heatmap was created using the gplots package as part of the R statistical computing environment. DHT is an abbreviation of dihydrotestosterone; RNAiAR, cells transfected with siRNA targeting the AR.

FIG. 2 is a bar graph showing cell viability in LNCaP cells grown in normal serum for 96 hours after RNAi-mediated suppression of individual androgen-independent AR target genes. The median cell viability for all RNAi samples is indicated by the horizontal line. Genes whose suppression led to a decline in viability greater than one standard deviation below the median are shown. Others are shown as gray bars. NTCl/NTC2 is an abbreviation for non-targeted control RNAi samples; AR signifies an AR RNAi positive control sample.

FIG. 3 is a bar graph showing expression of the indicated genes in LNCaP or Abl cells transfected with siRNA targeting the AR (RNAiAR) or a non-targeted control (NTC) detected by quantitative real-time PCR.

FIG. 4A is a plot showing prostate cancer relapse-free survival calculated with the log-rank test for 131 localized prostate cancer patients treated with primary therapy. The plot compares patients in the top decile with regard to level of expression of TPX2 (TPX2 Altered) with the remaining samples (TPX2 not altered.) For the log-rank test, p<10⁻⁷

FIG. 4B is a plot showing p-free survival calculated with the log-rank test for 131 localized prostate cancer patients treated with primary therapy. The plot compares patients in the top decile with regard to level of expression of KIF11 (KIF11 Altered) with the remaining samples (KIF11 not altered.)

### SEQUENCE LISTING

SEQ ID NO: 1 is a nucleic acid sequence of human ZWILCH.

SEQ ID NO: 2 is a nucleic acid sequence of human PTTG1.

SEQ ID NO: 3 is a nucleic acid sequence of human DEPDC1.

SEQ ID NO: 4 is a nucleic acid sequence of human TPX2.

SEQ ID NO: 5 is a nucleic acid sequence of human CDCA3.

SEQ ID NO: 6 is a nucleic acid sequence of human BCCIP.

SEQ ID NO: 7 is a nucleic acid sequence of human HMGB2.

SEQ ID NO: 8 is a nucleic acid sequence of human AURKB.

SEQ ID NO: 9 is a nucleic acid sequence of human KPNA2.

SEQ ID NO: 10 is a nucleic acid sequence of human AHCTF1.

SEQ ID NO: 11 is a nucleic acid sequence of human MYC.

SEQ ID NO: 12 is a nucleic acid sequence of human MCM7.

SEQ ID NO: 13 is a nucleic acid sequence of human DBF4.

SEQ ID NO: 14 is a nucleic acid sequence of human CDCA8.

SEQ ID NO: 15 is a nucleic acid sequence of human BARD1.

SEQ ID NO: 16 is a nucleic acid sequence of human SGOL2.

SEQ ID NO: 17 is a nucleic acid sequence of human CDC20.

SEQ ID NO: 18 is a nucleic acid sequence of human BUB3.

SEQ ID NO: 19 is a nucleic acid sequence of human DNM2.

SEQ ID NO: 20 is a nucleic acid sequence of human KIF11.

SEQ ID NO: 21 is a nucleic acid sequence of human androgen receptor (AR.)

### DETAILED DESCRIPTION

### I. Abbreviations

ADT androgen deprivation therapy

AR androgen receptor

CDC20 cell division cycle 20 homolog

CDCA3 cell division cycle associated 3

ChIP chromatin immunoprecipitation

CRPC castration resistant prostate cancer

CSPC castration sensitive prostate cancer

DEPDC1 DEP domain containing 1

DHT dihydrotestosterone

HMGB2 high-mobility group box 2

KIF 11 kinesin family member 11

MYC v-myc myelocytomatosis

PSA prostate specific antigen

QRTPCR quantitative real-time polymerase chain reaction

TPX2 TPX2, microtubule-associated, homolog

ZWILCH Zwilch, kinetochore associated, homolog

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Androgen receptor (AR): Also known as NR3C4, dihydrotestosterone receptor, or SBMA. A member of subfamily 3C (along with the glucocorticoid receptor, mineralocorticoid receptor, and progesterone receptor) of the nuclear receptor superfamily. The AR binds directly to DNA and modulates gene transcription upon binding of ligand (such as testosterone or dihydrotestosterone (DHT)). The AR also acts through direct protein-protein interactions, for example with other transcription factors or signal transduction proteins to modulate gene expression.

In one example, AR includes a full-length wild-type (or native) sequence, as well as AR allelic variants that retain at least one activity of an AR (such as ligand binding or DNA binding). In certain examples, AR has at least 80% sequence identity, for example at least 85%, 90%, 95%, or 98% sequence identity to SEQ ID NO: 21.

Antibody: A polypeptide including at least a light chain or heavy chain immunoglobulin variable region which specifically recognizes and binds an epitope of an antigen, such as a cancer survival factor-associated molecule or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. In some examples, antibodies of the present disclosure include those that are specific for TPX2, KIF11, ZWILCH, MYC, DEPDC1, CDCA3, HMGB2, or CDC20.

The term antibody includes intact immunoglobulins, as well the variants and portions thereof, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Array: An arrangement of molecules, such as biological macromolecules (such as peptides, antibodies, or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules (“features”) makes it possible to carry out a large number of analyses on a sample at one time. In certain example arrays, one or more molecules (such as an oligonucleotide probe) will occur on the array a plurality of times (such as two or three times), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes at least one (such as 1, 2, 3, 4, 5, 6, 7, or 8) oligonucleotide probes or primers which can be used to detect genes disclosed herein, such as TPX2, KIF11, ZWILCH, MYC, DEPDC1, CDCA3, HMGB2, or CDC20.

Protein-based arrays include probe molecules that are or include proteins (for example, antibodies), or where the target molecules are or include proteins, and arrays including nucleic acids to which proteins are bound, or vice versa. In some examples, an array contains one or more (such as 1, 2, 3, 4, 5, 6, 7, or 8) antibodies specific for one of TPX2, KIF11, ZWILCH, MYC, DEPDC1, CDCA3, HMGB2, and CDC20.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

In some examples, the array includes positive controls, negative controls, or both, for example molecules specific for detecting β-actin, 18S RNA, beta-micro globulin, glyceraldehyde-3-phosphate-dehydrogenase (GAPDH), and other housekeeping genes. In one example, the array includes 1 to 20 controls, such as 1 to 10 or 1 to 5 controls.

Binding or stable binding: An association between two substances or molecules, such as the association of an antibody with a polypeptide (such as a TPX2, KIF11, ZWILCH, MYC, DEPDC1, CDCA3, HMGB2, or CDC20 polypeptides), or a nucleic acid to another nucleic acid (such as the binding of an oligonucleotide probe to TPX2, KIF11, ZWILCH, MYC, DEPDC1, CDCA3, HMGB2, or CDC20 RNA or TPX2, KIF11, ZWILCH, MYC, DEPDC1, CDCA3, HMGB2, or CDC20 cDNA). Binding can be detected by any procedure known to one skilled in the art.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is an increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Biomarker: Molecular, biological or physical attributes that characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination.

A biomarker may be represented by the sequence of a nucleic acid from which it can be derived or any other chemical structure. Examples of such nucleic acids include miRNA, tRNA, siRNA, mRNA, cDNA, or genomic DNA sequences including any complimentary sequences thereof.

One example of a biomarker is a gene product, such as a protein or RNA molecule encoded by a particular DNA sequence. Expression of the gene product in a sample comprising prostate cancer cells signifies a particular outcome from the prostate cancer. One further example is any expression product of the TPX2, KIF11, ZWILCH, MYC, DEPDC1, CDCA3, HMGB2, or CDC20 gene.

Cancer: A malignant neoplasm that has undergone characteristic anaplasia with loss of differentiation, increased rate of growth, invasion of surrounding tissue, and is capable of metastasis. For example, prostate cancer is a malignant neoplasm that arises in or from prostate tissue.

Residual cancer is cancer that remains in a subject after any form of treatment given to the subject to reduce or eradicate cancer. Metastatic cancer is a cancer at one or more sites in the body other than the site of origin of the original (primary) cancer from which the metastatic cancer is derived. Local recurrence is reoccurrence of the cancer at or near the same site (such as in the same tissue) as the original cancer.

cDNA (complementary DNA): A piece of DNA lacking internal, non-coding segments (introns) and regulatory sequences which determine transcription. cDNA can be synthesized by reverse transcription from messenger RNA (mRNA) extracted from cells, for example TPX2, KIF11, ZWILCH, MYC, DEPDC1, CDCA3, HMGB2, or CDC20 cDNA reverse transcribed from TPX2, KIF11, ZWILCH, MYC, DEPDC1, CDCA3, HMGB2, or CDC20 mRNA. The amount of TPX2, KIF11, ZWILCH, MYC, DEPDC1, CDCA3, HMGB2, or CDC20 cDNA reverse transcribed from TPX2, KIF11, ZWILCH, MYC, DEPDC1, CDCA3, HMGB2, or CDC20 mRNA can be used to determine the amount of TPX2, KIF11, ZWILCH, MYC, DEPDC1, CDCA3, HMGB2, or CDC20 mRNA present in a biological sample and thus the amount of expression of TPX2, KIF11, ZWILCH, MYC, DEPDC1, CDCA3, HMGB2, or CDC20.

Cell division cycle 20 homolog (CDC20): A protein involved in regulation of cell division. One function of CDC20 is activation of the anaphase-promoting complex, which initiates chromatid separation and entrance into anaphase. CDC20 is also part of the spindle assembly checkpoint, which ensures that anaphase proceeds only when centromeres of all sister chromatids are lined up on the metaphase plate and attached to microtubules.

In one example, CDC20 includes a full-length wild-type (or native) sequence, as well as CDC20 allelic variants that retain the ability to be expressed at increased levels in a tumor, such as a prostate tumor. In certain examples, CDC20 has at least 80% sequence identity, for example at least 85%, 90%, 95%, or 98% sequence identity to SEQ ID NO: 17

Cell division cycle associated 3 (CDCA3): Also known as trigger of mitotic entry 1 (TOMEI). CDCA3 is a G 1 substrate of the anaphase-promoting complex. CDCA3 associates with Skp 1 and is required for degradation of Cdk1 inhibitory tyrosine kinase Weet Nucleic acid and protein sequences for CDCA3 are publicly available.

In one example, CDCA3 includes a full-length wild-type (or native) sequence, as well as CDCA3 allelic variants that retain the ability to be expressed at increased levels in a tumor, such as a prostate tumor. In certain examples, CDCA3 has at least 80% sequence identity, for example at least 85%, 90%, 95%, or 98% sequence identity to SEQ ID NO: 5.

Contacting: Placement in direct physical association; includes solid, liquid, and gaseous associations. Contacting includes contact between one molecule and another molecule. Contacting can occur in vitro with isolated cells or tissue or in vivo by administering to a subject, such as the administration of a treatment for Alzheimer's disease to a subject. The concept of contacting may also be encompassed by adding a molecule to a solid, liquid, or gaseous mixture.

Control: A reference standard. A control can be a known value indicative of basal expression of a gene, for example the amount of TPX2, KIF11, ZWILCH, MYC, DEPDC1, CDCA3, HMGB2, or CDC20 expressed in cells from a prostate cancer. A difference between the expression in a test sample (such as a biological sample obtained from a subject can be indicative of a biological state such as a particular disease outcome. For example, expression of TPX2, KIF11, ZWILCH, MYC, DEPDC1, CDCA3, HMGB2, or CDC20 in a prostate cancer sample greater than that of a control may be indicative of shorter survival time of the subject from which the prostate cancer sample was derived.

A may be any sample or standard used for comparison with an experimental sample. In some embodiments, the control is a sample obtained from a healthy patient or a non-tumor tissue sample obtained from a patient diagnosed with cancer (such as non-tumor tissue adjacent to the tumor). In some embodiments, the control is a historical control or standard reference value or range of values (such as a previously tested control sample, such as a group of cancer patients with poor prognosis, or group of samples that represent baseline or normal values, such as the level of one or more of the genes disclosed herein in non-tumor tissue). A control may also serve as a threshold level of expression of a biomarker that indicates a particular disease outcome.

DEP domain containing 1 (DEPDC1): A gene that is highly expressed in bladder cancer. DEPDC1 interacts with the zinc finger transcription factor ZNF224. Nucleic acid and protein sequences for DEPDCI are publicly available.

In one example, DEPDCI includes a full-length wild-type (or native) sequence, as well as DEPDCI allelic variants that retain the ability to be expressed at increased levels in a tumor, such as a prostate tumor. In certain examples, DEPDC1 has at least 80% sequence identity, for example at least 85%, 90%, 95%, or 98% sequence identity to SEQ ID NO: 3.

Detecting expression of a gene: Detection of a level of expression in either a qualitative or quantitative manner, for example by detecting nucleic acid or protein (such as a TPX2, KIF11, ZWILCH, MYC, DEPDC1, CDCA3, HMGB2, or CDC20 nucleic acid or protein) by routine methods known in the art or by any method yet to be disclosed in the art.

Differential expression or altered expression: A difference in the amount of messenger RNA, the conversion of mRNA to a protein, or both between two different samples. In some examples, the difference is relative to a control or threshold level of expression, such as an amount of gene expression in non-cancerous prostate tissue from.

DNA (deoxyribonucleic acid): A long chain polymer which includes the genetic material of most living organisms (some viruses have genes including ribonucleic acid, RNA.) The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Expression: The process by which the coded information of a gene is converted into an operational, non-operational, or structural part of a cell, such as the synthesis of an RNA or protein. Gene expression can be influenced by external signals. For instance, exposure of a cell to a hormone may stimulate expression of a hormone induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. In an example, gene expression can be monitored to determine the prognosis of a subject with a tumor (such as a prostate tumor), such as to predict a subject's survival or likelihood to develop metastasis.

The expression of a nucleic acid molecule in a test sample can be altered relative to a control sample, such as a normal or non-tumor sample. Alterations in gene expression, such as differential expression, include but are not limited to: (1) overexpression; (2) underexpression; or (3) suppression of expression. Alterations in the expression of a nucleic acid molecule can be associated with, and in fact cause, a change in expression of the corresponding protein.

Protein expression can also be altered in some manner to be different from the expression of the protein in a normal (e.g., non-tumor) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Controls or standards for comparison to a sample, for the determination of differential expression, include samples believed to be normal (in that they are not altered for the desired characteristic, for example a sample from a subject who does not have cancer, such as prostate cancer) as well as laboratory values (e.g., a range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values.

High-mobility group box 2 (HMGB2): Also known as high-mobility group protein 2—a member of the non-histone chromosomal high mobility group protein family. These proteins are associated with chromatin and are able to bend DNA and form DNA circles. Nucleic acid and protein sequences for HMGB2 are publicly available. In one example, HMGB2 includes a full-length wild-type (or native) sequence, as well as HMGB2 allelic variants that retain the ability to be expressed at increased levels in a tumor, such as a prostate tumor. In certain examples, HMGB2 has at least 80% sequence identity, for example at least 85%, 90%, 95%, or 98% sequence identity to SEQ ID NO: 7.

Hybridization: To form base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 60% identity)

Hybridization: 6×SSC at RT to 55° C. for 16-20 hours

Wash at least twice: 2×-3×SSC at RT to 55° C. for 20-30 minutes each

Isolated: An “isolated” biological component (such as a nucleic acid molecule, protein or organelle) has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Kinesin family member 11 (KIF11): Also known as TR-interacting protein 5, kinesin-like protein 1, kinesin-related motor protein Eg5, and thyroid receptor interacting protein 5. KIF11 is a member of the family of kinesin-like motor proteins, involved in spindle dynamics. KIF11 is involved in chromosome positioning, centromere separation, and establishing a bipolar spindle during mitosis.

Nucleic acid and protein sequences for KIF11 are publicly available. In one example, KIF11 includes a full-length wild-type (or native) sequence, as well as KIF11 allelic variants that retain the ability to be expressed at increased levels in a tumor, such as a prostate tumor. In certain examples, KIF11 has at least 80% sequence identity, for example at least 85%, 90%, 95%, or 98% sequence identity to SEQ ID NO: 20.

Label: A detectable compound or composition that is conjugated directly or indirectly to another molecule to facilitate detection of that molecule. Specific, non-limiting examples of labels include radioactive isotopes, enzyme substrates, co-factors, ligands, chemiluminescent or fluorescent agents, haptens, and enzymes. In some examples, a label is attached to an antibody or nucleic acid to facilitate detection of the molecule that the antibody or nucleic acid specifically binds, such as a TPX2, KIF11, ZWILCH, MYC, DEPDC1, CDCA3, HMGB2, or CDC20 protein or nucleic acid.

v-myc myelocytomatosis viral oncogene homolog (MYC): A protooncogene MYC of a transcription factor network that regulates cellular proliferation, replicative potential, growth, differentiation, and apoptosis. Nucleic acid and protein sequences for MYC are publicly available. In one example, MYC includes a full-length wild-type (or native) sequence, as well as MYC allelic variants that retain the ability to be expressed at increased levels in a tumor, such as a prostate tumor. In certain examples, MYC has at least 80% sequence identity, for example at least 85%, 90%, 95%, or 98% sequence identity to SEQ ID NO: 11.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear. A nucleic acid molecule may also be termed a polynucleotide and the terms are used interchangeably.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides.

An oligonucleotide probe is an oligonucleotide that is used to detect the presence of a complementary sequence by molecular hybridization. In particular examples, oligonucleotide probes include a label that permits detection of oligonucleotide probe:target sequence hybridization complexes. In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

An oligonucleotide primer is an oligonucleotide that is used to prime a nucleic acid amplification. An oligonucleotide primer can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule.

The specificity of an oligonucleotide primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure (for example, to amplify all or any part of TPX2, KIF11, ZWILCH, MYC, DEPDC1, CDCA3, HMGB2, or CDC20) include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-50 nucleotides, 20-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

Nucleic acid probes and/or primers can be readily prepared based on the nucleic acid molecules provided herein. PCR primer pairs and probes can be derived from a known sequence for example, by using any of a number of computer programs intended for that purpose such as Primer (Version 0.5, ©1991, Whitehead Institute for Biomedical Research, Cambridge, Mass.) or PRIMER EXPRESS® Software (Applied Biosystems, AB, Foster City, Calif.).

Methods for preparing and using oligonucleotide and other nucleic acid probes and primers and methods for labeling and guidance in the choice of labels appropriate for various purposes are described, for example, in Sambrook et al (In Molecular Cloning: A Laboratory Manual, CSHL, New York, 1989), Ausubel et al (ed.) (In Current Protocols in Molecular Biology, John Wiley & Sons, New York, 1998), and Innis et al (PCR Protocols, A Guide to Methods and Applications, Academic Press, Inc., San Diego, Calif., 1990).

Polypeptide: a polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Prognosis: A prediction of the course of a disease, such as cancer (for example, prostate cancer). The prediction can include determining the likelihood of a subject to develop aggressive, recurrent disease, to develop one or more metastases, to survive a particular amount of time (e.g., determine the likelihood that a subject will survive 3 months, 6 months, 1, 2, 3, 4, or 5 years), to respond to a particular therapy (e.g., hormone therapy), or combinations thereof.

Prostate cancer: A malignant tumor, generally of glandular origin, of the prostate. In some examples, prostate cancer includes an adenocarcinoma, transitional cell carcinoma, squamous cell carcinoma, sarcoma, or small cell carcinoma of the prostate. In other examples, prostate cancer includes metastatic prostate cancer, for example metastasis of a prostate tumor to another tissue or organ, such as lung, bone, liver, or brain.

Sample (or biological sample): A specimen containing genomic DNA, RNA (including mRNA), protein, or combinations thereof, obtained from a subject. As used herein, biological samples include cells, tissues, and bodily fluids, such as: blood;

derivatives and fractions of blood, such as plasma or serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; or bone marrow aspirates. In a particular example, a sample includes a tumor biopsy (such as a prostate tumor biopsy). In another example, a sample includes circulating tumor cells, such as tumor cells present in blood of a subject with a tumor.

Obtaining a biological sample from a subject includes, but need not be limited to any method of collecting a particular sample known in the art. Obtaining a biological sample from a subject also encompasses receiving a sample that was collected at a different location than where a method is performed; receiving a sample that was collected by a different individual than an individual that performs the method, receiving a sample that was collected at any time period prior to the performance of the method, receiving a sample that was collected using a different instrument than the instrument that performs the method, or any combination of these. Obtaining a biological sample from a subject also encompasses situations in which the collection of the sample and performance of the method are performed at the same location, by the same individual, at the same time, using the same instrument, or any combination of these.

A biological sample encompasses any fraction of a biological sample or any component of a biological sample that may be isolated and/or purified from the biological sample. For example: when cells are isolated from blood or tissue, including specific cell types sorted on the basis of biomarker expression; or when nucleic acid or protein is purified from a fluid or tissue; or when blood is separated into fractions such as plasma, serum, buffy coat PBMC's or other cellular and non-cellular fractions on the basis of centrifugation and/or filtration. A biological sample further encompasses biological samples or fractions or components thereof that have undergone a transformation of mater or any other manipulation. For example, a cDNA molecule made from reverse transcription of mRNA purified from a biological sample may be termed a biological sample.

Sensitivity and specificity: Statistical measurements of the performance of a binary classification test. Sensitivity measures the proportion of actual positives which are correctly identified (e.g., the percentage of tumors that are identified as having a poor prognosis). Specificity measures the proportion of negatives which are correctly identified (e.g., the percentage of tumors identified as not having a poor prognosis).

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer Appls in the Biosciences 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a nucleic acid sequence of TPX2, KIF11, ZWILCH, MYC, DEPDC1, CDCA3, HMGB2, or CDC20.

Specific Binding Agent: An agent that binds substantially or preferentially only to a defined target such as a protein, enzyme, polysaccharide, oligonucleotide, DNA, RNA, recombinant vector or a small molecule. In an example, a “specific binding agent” is capable of binding to a TPX2, KIF11, ZWILCH, MYC, DEPDC1, CDCA3, HMGB2, or CDC20 gene product, such as a TPX2, KIF11, ZWILCH, MYC, DEPDC1, CDCA3, HMGB2, or CDC20 mRNA, cDNA, or protein. Thus, a nucleic acid-specific binding agent binds substantially only to the defined nucleic acid, such as RNA, or to a specific region within the nucleic acid.

A protein-specific binding agent binds substantially only the defined protein, or to a specific region within the protein. For example, a specific binding agent includes antibodies and other agents that bind substantially to a specified polypeptide, for example a specific binding agent that specifically binds TPX2, KIF11, ZWILCH, MYC, DEPDC1, CDCA3, HMGB2, or CDC20, can be an antibody, for example a monoclonal or polyclonal antibody or a ligand for TPX2, KIF11, ZWILCH, MYC, DEPDC1, CDCA3, HMGB2, or CDC20. Antibodies can be monoclonal or polyclonal antibodies that are specific for the polypeptide as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999). A specific binding agent that binds to a particular biomarker may also be called a specific binding reagent. These terms may be used interchangeably.

Subject: Multi-cellular vertebrate organism, a category that includes human and non-human mammals.

Survival: Time interval between date of diagnosis or first treatment (such as surgery or first treatment) and a specified event, such as development of resistance to a particular therapy, relapse, metastasis or death. Overall survival is the time interval between the date of diagnosis or first treatment and date of death or date of last follow up. Relapse-free survival is the time interval between the date of diagnosis or first treatment and date of a diagnosed relapse (such as a locoregional recurrence) or date of last follow up. Metastasis-free survival is the time interval between the date of diagnosis or first treatment and the date of diagnosis of a metastasis or date of last follow up.

TPX2, microtubule-associated, homolog (Xenopus laevis) (TPX2): Also known as protein f1s353; hepatocellular carcinoma-associated antigen 519; restricted expression proliferation-associated protein 100; and targeting protein for Xklp2. TPX2 is a component of the spindle apparatus and interacts with Aurora-A serine-threonine kinase.

Nucleic acid and protein sequences for TPX2 are publicly available. In one example, TPX2 includes a full-length wild-type (or native) sequence, as well as TPX2 allelic variants that retain the ability to be expressed at increased levels in a tumor, such as a prostate tumor. In certain examples, TPX2 has at least 80% sequence identity, for example at least 85%, 90%, 95%, or 98% sequence identity to SEQ ID NO: 4.

Zwilch, kinetochore associated, homolog (ZWILCH): A component of the mitotic checkpoint, which prevents cells from prematurely exiting mitosis. ZWILCH is targeted to the kinetochores during mitosis. Nucleic acid and protein sequences for ZWILCH are publicly available.

In one example, ZWILCH includes a full-length wild-type (or native) sequence, as well as ZWILCH allelic variants that retain the ability to be expressed at increased levels in a tumor, such as a prostate tumor. In certain examples, ZWILCH has at least 80% sequence identity, for example at least 85%, 90%, 95%, or 98% sequence identity to SEQ ID NO: 1.

III. Methods of Determining Prognosis of a Subject with Cancer

Disclosed herein are gene expression profiles that can be used to determine the prognosis in subjects with cancer (such as prostate cancer). In some examples, determining the prognosis includes predicting the outcome (such as chance of tumor recurrence, metastasis, or survival) of the subject with a tumor. In other examples, determining the prognosis includes predicting whether the tumor is or is likely to become resistant to a therapy (such as chemotherapy or hormone therapy). Thus, provided herein are methods of prognosing a subject with a tumor (such as a prostate tumor).

In some embodiments, the methods include detecting expression of one or more (such as 1, 2, 3, 4, 5, 6, 7, or all) gene products of TPX2, KIF11, ZWILCH, MYC, DEPDC1, CDCA3, HMGB2, and CDC20 in a sample from the subject, and comparing expression of the one or more genes in the sample to a threshold level of expression. In some examples, the methods include detecting expression of five or more (such as 5, 6, 7, or all) gene products of TPX2, KIF11, ZWILCH, MYC, DEPDCI, CDCA3, HMGB2, and CDC20. In other examples, the method includes detecting expression of one or more (such as 1, 2, 3, 4, 5, 6, 7, 8, 9, 10, 11, 12, 13, 14, 15, 16, 17, 18, 19, or all) products of the genes disclosed in Table 1. In some embodiments of the method, expression of one or more (such as 1, 2, 3, 4, 5, 6, 7, or all) gene products of TPX2, KIF11, ZWILCH, MYC, DEPDC1, CDCA3, HMGB2, and CDC20 in a sample that exceeds a threshold level of expression indicates a poor prognosis, such as a decreased chance of survival (for example decreased overall survival, relapse-free survival, or metastasis-free survival) or resistance or likelihood to develop resistance to a therapy (such as hormone therapy, for example, ADT for prostate cancer). In particular examples, expression of five or more (such as 5, 6, 7, or all) of TPX2, KIF11, ZWILCH, MYC, DEPDC1, CDCA3, HMGB2, and CDC20 in the sample that exceeds a threshold level of expression indicates a poor prognosis, such as a decreased chance of survival (for example decreased overall survival, relapse-free survival, or metastasis free survival) or resistance or likelihood to develop resistance to a therapy (such as hormone therapy, for example, ADT for prostate cancer).

In one an example, a decreased overall survival includes a survival time equal to or less than 60 months, such as 50 months, 40 months, 30 months, 20 months, 12 months, 6 months, or 3 months from time of diagnosis or first treatment. In another example, decreased relapse-free survival includes a relapse-free period equal to or less than 60 months, such as 50 months, 40 months, 30 months, 20 months, 12 months, 6 months, or 3 months from time of diagnosis or first treatment. In further examples, decreased metastasis-free survival includes a metastasis-free period equal to or less than 60 months, such as 50 months, 40 months, 30 months, 20 months, 12 months, 6 months, or 3 months from time of diagnosis or first treatment.

In additional examples, resistance to a therapy (such as chemotherapy or hormone therapy) includes a tumor that does not respond to an initial or subsequent treatment. A condition that does not respond to an initial treatment is referred to as having intrinsic resistance. A condition that responds to an initial therapy treatment, but does not respond to a subsequent treatment with the same therapy is referred to as having acquired resistance. In some examples, a poor prognosis includes current tumor resistance to a therapy (such as hormone therapy). In other examples, a poor prognosis includes developing tumor resistance to a therapy (such as hormone therapy) in a period equal to or less than 72 months, 60 months, such as 50 months, 40 months, 30 months, 24 months, 18 months, 12 months, 6 months, or 3 months from time of diagnosis or first treatment. In some examples, the tumor is a prostate tumor that has or is likely to acquire resistance to hormone therapy (such as androgen deprivation therapy; ADT).

ADT (or androgen suppression therapy) can include treatment with luteinizing hormone-releasing hormone (LHRH) agonists or analogs (for example, leuprolide, goserelin, triptorelin, buserelin, or histrelin), LHRH antagonists (for example, abarelix or degarelix), antiandrogens (for example, flutamide, bicalutamide, or nilutamide), ketoconazole, or a combination of two or more thereof. In particular examples, the tumor is or is likely to acquire resistance to an LHRH agonist (such as leuprolide or goserelin) or surgical removal of the testes. Resistance to hormone therapy can be determined by one of skill in the art, for example by observing increasing PSA levels over time, despite a castrate level of testosterone in the serum.

Expression of the disclosed genes can be detected and/or quantified using any suitable methodology known in the art or yet to be disclosed. For example, detection of gene expression can be accomplished by detecting nucleic acid molecules (such as RNA) using nucleic acid amplification methods (such as RT-PCR) or array analysis. Detection of gene expression can also be accomplished using immunoassays that detect proteins (such as ELISA, Western blot, or RIA assay). Additional methods of detecting gene expression are well known in the art and are described in greater detail below.

In one example, expression of the disclosed genes is detected and/or quantified in a biological sample. In a particular example, the biological sample is a tumor sample, such as a tumor biopsy (for example, a prostate tumor biopsy). In some examples, a tumor sample includes tumor tissue that is unfixed, frozen, fixed in formalin and/or embedded in paraffin. In another example, the sample is a peripheral blood sample, such as a sample including circulating tumor cells. In other examples, the sample is urine, saliva, cerebrospinal fluid, prostate fluid, pus, or bone marrow aspirate.

The altered expression of the disclosed genes associated with tumor prognosis can be any quantity of expression that is correlated with a poor prognosis. In some embodiments, the increase or decrease in expression is at least 1.5-fold, at least 2-fold, at least 2.5-fold, at least 3-fold, at least 4-fold, at least 5-fold, at least 7-fold, at least 10-fold, at least 15-fold, at least 20-fold, or more relative to a threshold level of expression.

A threshold level of expression is a quantified level of expression of a particular gene or set of genes. An expression level of a gene or set of genes in a sample that exceeds or falls below the threshold level of expression is predictive of a particular disease state or outcome. In but one example (simplified for ease of explanation) expression of TPX2 exceeding a threshold level of expression is predictive of disease relapse in patients with prostate cancer.

The nature and numerical value (if any) of the threshold level of expression will vary based on the method chosen to determine the expression the gene or gene set used in the prediction. In light of this disclosure, any person of skill in the art would be capable of determining the threshold level of TPX2 expression in a patient sample that would be predictive of reduced survival in prostate cancer using any method of measuring specific RNA or protein expression now known in the art or yet to be disclosed.

The concept of a threshold level of expression should not be limited to a single value or result. Rather, the concept of a threshold level of expression encompasses multiple threshold expression levels that could signify, for example, a high, medium, or low probability of, for example, disease free survival. Alternatively, there could be a low threshold of expression wherein expression of TPX2 in the sample below the threshold indicates that the subject is likely to have a good prognosis and a separate high threshold of expression wherein TPX2 expression in the sample above the threshold indicates that the subject has a poor prognosis. Expression in the sample that falls between the two threshold values is inconclusive as to whether the subject has or does not have a poor prognosis.

To obtain a threshold value of TPX2 expression that indicates that a subject has a poor outcome for a particular method of measuring TPX2 expression (for example, RTPCR, ELISA, ISH, or IHC) one would determine TPX2 expression using samples obtained from a first cohort of subjects known to have reduced survival in prostate cancer and from a second cohort known not to have reduced survival. TPX2 expression is determined in both cohorts and an expression profile of the desired expression that signifies that a subject has a poor prognosis. Preferably, the threshold level of expression will be the level of expression that provide the maximal ability to predict whether or not a subject has a poor prognosis and will maximize both the selectivity and sensitivity of the test. The predictive power a threshold level of expression may be evaluated by any of a number of statistical methods known in the art. One of skill in the art will understand which statistical method to select on the basis of the method of determining TPX2 expression and the data obtained. Examples of such statistical methods include:

Receiver Operating Characteristic curves, or “ROC” curves, may be calculated by plotting the value of a variable versus its relative frequency in each of two populations. Using the distribution, a threshold is selected. The area under the ROC curve is a measure of the probability that the expression correctly indicates the diagnosis. If the distribution of TPX2 expression between the two cohorts overlaps, then TPX2 expression values from subjects falling into the area of overlap then the subject providing the sample cannot be diagnosed. See, e.g., Hanley et al, Radiology 143, 29-36 (1982) hereby incorporated by reference in its entirety. In that case, a low threshold of expression and a high threshold of expression may be selected.

An odds ratio measures effect size and describes the amount of association or non-independence between two groups. An odds ratio is the ratio of the odds that TPX2 expression above the threshold will occur in samples from a cohort of subjects known to have or who go on to develop AD over the odds that TPX2 expression above the threshold will occur in samples from a cohort of subjects known not to have or who will not go on to develop AD. An odds ratio of 1 indicates that TPX2 expression above the threshold is equally likely in both cohorts. An odds ratio greater or less than 1 indicates that expression of the marker is more likely to occur in one cohort or the other.

A hazard ratio may be calculated by estimate of relative risk. Relative risk is the chance that a particular event will take place. For example: a relative risk may be calculated from the ratio of the probability that samples that exceed a threshold level of expression of TPX2 will be from patients that have a poor prognosis over the probability that samples that do not exceed the threshold will be from patients that do not have a poor prognosis. In the case of a hazard ratio, a value of 1 indicates that the relative risk is equal in both the first and second groups and that the assay has little or no predictive value; a value greater or less than 1 indicates that the risk is greater in one group or another, depending on the inputs into the calculation.

Multiple threshold levels of expression may be selected by so-called “tertile,” “quartile,” or “quintile” analyses. In these methods, multiple groups can be considered together as a single population, and are divided into 3 or more bins having equal numbers of individuals. The boundary between two of these “bins” may be considered threshold levels of expression indicating a particular level of risk that the subject has or will have a poor prognosis. A risk may be assigned based on which “bin” a test subject falls into.

The threshold level of expression may also differ based on the purpose of the test. For a test to determine whether or not a subject has or does not a poor prognosis, two cohorts of subjects may be tested: one cohort of subjects known to have a poor prognosis, and another known not to have a poor prognosis. TPX2 expression is determined by the same method in both cohorts, and the threshold level of expression to differentiate the cohorts is determined.

One type of threshold level of expression is the amount or valuation of expression relative to one or more controls or standards. Expression may be above or below a control that is known to be equivalent to the threshold level of expression. The control may be any suitable control against which to compare expression of a gene in a sample. In some embodiments, the control sample is non-tumor tissue. In some examples, the non-tumor tissue is obtained from the same subject, such as non-tumor tissue that is adjacent to the tumor. In other examples, the non-tumor tissue is obtained from a healthy control subject. In other examples, a set of controls that are equivalent to known expression levels are evaluated to formulate a standard curve. Expression in the sample is then quantified on the basis of that standard curve and then compared to the threshold level of expression.

In some embodiments, the disclosed methods further include determining additional indicators of prognosis for the subject. In specific examples, the tumor is a prostate tumor, and the methods include measuring the level of prostate specific antigen (PSA) of the subject. Methods of measuring PSA levels of a subject (such as in a sample from the subject, for example a blood sample) are known to one of skill in the art and include immunoassays (such as electrochemiluminescent immunoassay). In some instances, the subject has a PSA level higher than a normal PSA level (for example, higher than 4 ng/mL, such as about 4-50 ng/mL, about 4-10 ng/mL, or about 10-25 ng/mL). In some examples, an increased (higher than normal) PSA level indicates that the subject has a poor prognosis. In one example, a PSA level of 10.0 or greater indicates that the subject has a poor prognosis. PSA levels can vary based on the age and health status of the subject. One of skill in the art can determine a normal or abnormal PSA level in a subject.

In other examples, the tumor is a prostate tumor and the methods include detecting the presence of a TMPRSS2-ERG gene fusion in the sample from the subject. Methods of detecting a TMPRSS2-ERG gene fusion are known to one of skill in the art and include in situ hybridization (for example, fluorescent in situ hybridization or colorimetric in situ hybridization), Southern blot, Northern blot, polymerase chain reaction (such as reverse transcription PCR), Western blot, or immunohistochemistry. In some examples, presence of TMPRSS2-ERG gene fusion indicates that the subject has a poor prognosis.

The disclosed methods can be used to determine the prognosis of a subject with cancer. In a particular example, cancer includes prostate cancer.

### IV. Detecting Gene Expression

A. Detection of Nucleic Acids

Expression of a nucleic acid in a sample can be detected using routine methods.

In some examples, nucleic acids in a biological sample are isolated, amplified, or both. In some examples, amplification and detection of expression occur simultaneously or nearly simultaneously. For example, nucleic acids can be isolated and amplified by employing commercially available kits. In an example, the biological sample can be incubated with primers that permit the amplification of mRNA of at least one of TPX2, KIF11, ZWILCH, MYC, DEPDC1, CDCA3, HMGB2, and/or CDC20, under conditions sufficient to permit amplification of such products.

Methods of determining the amount of nucleic acids, such as mRNA encoding TPX2, KIF11, ZWILCH, MYC, DEPDC1, CDCA3, HMGB2, and/or CDC20 based on hybridization analysis and/or sequencing are known in the art. Methods known in the art for the quantification of mRNA expression in a sample include northern blotting and in situ hybridization (Parker & Barnes, Methods in Molecular Biology 106 247-283 (1999); RNAse protection assays (Hod, Biotechniques 13, 852-854 (1992)); and PCR-based methods, such as reverse transcription polymerase chain reaction (RT-PCR) (Weis et al., Trends in Genetics 8, 263-264 (1992)). Representative methods for sequencing-based gene expression analysis include Serial Analysis of Gene Expression (SAGE), and gene expression analysis by massively parallel signature sequencing (MPSS). (See Mardis ER, Annu. Rev. Genomics Hum Genet 9, 387-402 (2008)). In some embodiments, determining the amount of TPX2, KIF11, ZWILCH, MYC, DEPDC1, CDCA3, HMGB2, and/or CDC20 expressed in a biological sample includes determining the amount of TPX2, KIF11, ZWILCH, MYC, DEPDC1, CDCA3, HMGB2, and/or CDC20 mRNA in the biological sample.

Methods for quantifying mRNA are well known in the art. In one example, the method utilizes reverse transcriptase polymerase chain reaction (RT-PCR). Generally, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT,) though any enzyme or fragment thereof capable of synthesizing cDNA from an RNA template may be used. The reverse transcription step is typically primed using specific primers, random hexamers, or oligo-dT primers, depending on the circumstances and the goal of expression profiling. For example, extracted RNA can be reverse-transcribed using a GENEAMP® RNA PCR kit (Perkin Elmer, Calif., USA), following the manufacturer's instructions. The derived cDNA can then be used as a template in the subsequent PCR reaction.

Although the PCR step can use any of a number of thermostable DNA-dependent DNA polymerases, it typically employs a Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data. Examples of fluorescent labels that may be used in quantitative PCR include but need not be limited to: HEX, TET, 6-FAM, JOE, Cy3, Cy5, ROX TAMRA, and Texas Red. Examples of quenchers that may be used in quantitative PCR include, but need not be limited to TAMRA (which may be used as a quencher with HEX, TET, or 6-FAM), BHQ1, BHQ2, or DABCYL.

TAQMAN® RT-PCR can be performed using commercially available equipment, such as, for example, ABI PRISM 7700® Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700® Sequence Detection System. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are the mRNA products of housekeeping genes.

Additionally, quantitative PCR may be performed upon a cDNA resulting from the reverse transcription of a sample from a subject without the use of a labeled oligonucleotide probe that binds to a sequence between the primers. In some of these techniques, PCR amplification is tracked by the binding of a fluorescent dye such as SYBR green to the double stranded PCR product during the amplification reaction. SYBR green binds to double stranded DNA, but not to single stranded DNA. In addition, SYBR green fluoresces strongly at a wavelength of 497 nm when it is bound to double stranded DNA, but does not fluoresce when it is not bound to double stranded DNA. As a result, the intensity of fluorescence at 497 nm may be correlated with the amount of amplification product present at any time during the reaction. The rate of amplification may in turn be correlated with the amount of template sequence present in the initial sample. Generally, Ct values are calculated similarly to those calculated using the TaqMan® system. Because the probe is absent, amplification of the proper sequence may be checked by any of a number of techniques. One such technique involves running the amplification products on an agarose or other gel appropriate for resolving nucleic acid fragments and comparing the amplification products from the quantitative real time PCR reaction with control DNA fragments of known size.

An RNA expression level within a sample may be quantified in comparison to an internal standard such as a housekeeping gene. When housekeeping gene expression is determined in the same sample as, for example, TPX2, TPX2 expression may be normalized to the expression of the housekeeping gene. So expression of the housekeeping gene serves as an internal normalization control that serves to account for sample-to-sample variability in terms of total RNA present. A housekeeping gene may be any gene that is constitutively expressed in most or all tissues in an organism at a constant level of expression. See Eisenberg and Levanon, Trends in Genetics 19, 362-365 (2003.) A list of human housekeeping genes is available at http://www.compugen.co.il/supp_info/Housekeeping_genes.html, last checked 8 Mar. 2012. One of skill in the art would know how to select one or more acceptable housekeeping genes to be used in any method of assessing mRNA expression of a particular target gene.

In one embodiment, a nucleic acid sample is utilized, such as the total mRNA isolated from a biological sample. The biological sample can be from any biological tissue or fluid from the subject of interest, such as a subject who is suspected of having cardiovascular disease. Such samples include, but are not limited to, blood, blood cells (such as white blood cells) or tissue biopsies including spleen tissue.

Nucleic acids (such as mRNA) can be isolated from the sample according to any of a number of methods well known to those of skill in the art. Methods of isolating total mRNA are well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993). In one example, the total nucleic acid is isolated from a given sample using, for example, an acid guanidinium-phenol-chloroform extraction method, and polyA+ mRNA is isolated by oligo dT column chromatography or by using (dT)n magnetic beads (see, for example, Sambrook et al, Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, N.Y. (1987)). In another example, oligo-dT magnetic beads may be used to purify mRNA (Dynal Biotech Inc., Brown Deer, Wis.). Nucleic acid may be isolated from blood either by lysing cells in whole blood prior to nucleic acid isolation or it may be isolated from a fraction of whole blood, such as PBMC. The nucleic acid sample can be amplified prior to hybridization. If a quantitative result is desired, a method is utilized that maintains or controls for the relative frequencies of the amplified nucleic acids. Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. The array can then include probes specific to the internal standard for quantification of the amplified nucleic acid.

Primers and probes used in quantitative PCR may be oligonucleotides. Oligonucleotide synthesis is the chemical synthesis of oligonucleotides with a defined chemical structure and/or nucleic acid sequence by any method now known in the art or yet to be disclosed. Oligonucleotide synthesis may be carried out by the addition of nucleotide residues to the 5′-terminus of a growing chain. Elements of oligonucleotide synthesis include: De-blocking (detritylation): A DMT group is removed with a solution of an acid, such as TCA or Dichloroacetic acid (DCA), in an inert solvent (dichloromethane or toluene) and washed out, resulting in a free 5′ hydroxyl group on the first base. Coupling: A nucleoside phosphoramidite (or a mixture of several phosphoramidites) is activated by an acidic azole catalyst, tetrazole, 2-ethylthiotetrazole, 2-bezylthiotetrazole, 4,5-dicyanoimidazole, or a number of similar compounds. This mixture is brought in contact with the starting solid support (first coupling) or oligonucleotide precursor (following couplings) whose 5′-hydroxy group reacts with the activated phosphoramidite moiety of the incoming nucleoside phosphoramidite to form a phosphite triester linkage. The phosphoramidite coupling may be carried out in anhydrous acetonitrile. Unbound reagents and by-products may be removed by washing.

A small percentage of the solid support-bound 5′-OH groups (0.1 to 1%) remain unreacted and should be permanently blocked from further chain elongation to prevent the formation of oligonucleotides with an internal base deletion commonly referred to as (n-1) shortmers. This is done by acetylation of the unreacted 5′-hydroxy groups using a mixture of acetic anhydride and 1-methylimidazole as a catalyst. Excess reagents are removed by washing.

The newly formed tricoordinated phosphite triester linkage is of limited stability under the conditions of oligonucleotide synthesis. The treatment of the support-bound material with iodine and water in the presence of a weak base (pyridine, lutidine, or collidine) oxidizes the phosphite triester into a tetracoordinated phosphate triester, a protected precursor of the naturally occurring phosphate diester internucleosidic linkage. This step can be substituted with a sulfurization step to obtain oligonucleotide phosphorothioates. In the latter case, the sulfurization step is carried out prior to capping. Upon the completion of the chain assembly, the product may be released from the solid phase to solution, deprotected, and collected. Products may be isolated by HPLC to obtain the desired oligonucleotides in high purity.

In one embodiment, the hybridized nucleic acids are detected by detecting one or more labels attached to the sample nucleic acids. The labels can be incorporated by any of a number of methods. In one example, the label is simultaneously incorporated during the amplification step in the preparation of the sample nucleic acids. Thus, for example, polymerase chain reaction (PCR) with labeled primers or labeled nucleotides will provide a labeled amplification product. In one embodiment, transcription amplification, as described above, using a labeled nucleotide (such as fluorescein-labeled UTP and/or CTP) incorporates a label into the transcribed nucleic acids. Alternatively, a label may be added directly to the original nucleic acid sample (such as mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore). Detectable labels suitable for use include any composition detectable by spectroscopic, photochemical, biochemical, immunochemical, electrical, optical or chemical means. Useful labels include biotin for staining with labeled streptavidin conjugate, magnetic beads (for example DYNABEADS™), fluorescent dyes (for example, fluorescein, Texas red, rhodamine, green fluorescent protein, and the like), radiolabels (for example, ³H, ¹²⁵I, ³⁵S, ¹⁴C, or ³²P), enzymes (for example, horseradish peroxidase, alkaline phosphatase and others commonly used in an ELISA), and colorimetric labels such as colloidal gold or colored glass or plastic (for example, polystyrene, polypropylene, latex, etc.) beads. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149; and U.S. Pat. No. 4,366,241. Methods of detecting such labels are also well known. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tijssen, ed. Elsevier, N.Y., 1993).

Nucleic acid hybridization involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. These steps have been standardized for commercially available array systems.

Methods for evaluating the hybridization results vary with the nature of the specific probe nucleic acids used as well as the controls provided. In one embodiment, simple quantification of the fluorescence intensity for each probe is determined. This is accomplished simply by measuring probe signal strength at each location (representing a different probe) on the array (for example, where the label is a fluorescent label, detection of the amount of florescence (intensity) produced by a fixed excitation illumination at each location on the array). Comparison of the absolute intensities of an array hybridized to nucleic acids from a “test” sample (such as prostate cancer tissue from a subject with an unknown prognosis) with intensities produced by a “control” sample (such as normal prostate tissue from the same patient) provides a measure of the relative expression of the nucleic acids that hybridize to each of the probes.

B. Detection of Proteins

As an alternative to, or in addition to, detecting nucleic acids, proteins can be detected using routine methods such as Western blot, immunohistochemistry, ELISA, or mass spectrometry. In some examples, proteins are purified before detection. In one example, at least one of TPX2, KIF11, ZWILCH, MYC, DEPDC1, CDCA3, HMGB2, and CDC20 is detected by incubating the biological sample with an antibody that specifically binds to the protein. In another example, at least one of the genes disclosed in Table 1 is detected by incubating the biological sample with an antibody that specifically binds to the protein. The primary antibody can include a detectable label. For example, the primary antibody can be directly labeled, or the sample can be subsequently incubated with a secondary antibody that is labeled (for example with a fluorescent label). The label can then be detected, for example by microscopy, ELISA, flow cytometry, or spectrophotometry. In another example, the biological sample is analyzed by Western blotting for detecting expression of at least one of TPX2, KIF11, ZWILCH, MYC, DEPDC1, CDCA3, HMGB2, and CDC20, or at least one of the genes disclosed in Table 1.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Nonlimiting examples of suitable prosthetic group complexes include streptavidin:biotin and avidin:biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium and non-limiting exemplary radioactive labels include ¹²⁵I, ¹³¹I, ³⁵S or ³H.

Exemplary commercially available antibodies include TPX2 antibodies (such as catalog numbers sc-26275, sc-271570, and sc-26273, Santa Cruz Biotechnology, Santa Cruz, Calif.; catalog numbers ab32795 and ab71816, Abeam, Cambridge, Mass.), KIF11 antibodies (such as catalog numbers sc-31644 and sc-66872, Santa Cruz Biotechnology; catalog numbers ab37009 and ab37814, Abeam); ZWILCH antibodies (such as catalog numbers sc-66302 and sc-135615, Santa Cruz Biotechnology; catalog numbers ab101403 and ab57533, Abeam); MYC antibodies (such as catalog numbers sc-70468 and sc-70463, Santa Cruz Biotechnology); DEPDC1 antibodies (such as catalog numbers sc-164170 and sc-86115, Santa Cruz Biotechnology; catalog numbers ab57591 and ab76647, Abeam); CDCA3 antibodies (such as catalog number sc-134625, Santa Cruz Biotechnology; catalog numbers ab69608 and ab57795, Abeam); HMGB2 antibodies (such as catalog numbers sc-8758 and sc-271689, Santa Cruz Biotechnology; catalog numbers ab61169 and ab64861, Abcam); and CDC20 antibodies (such as catalog numbers ab26483, ab64877, and ab18217, Abcam). One of skill in the art can identify or produce other suitable antibodies.

In an alternative example, protein expression can be assayed in a biological sample by a competition immunoassay utilizing standards labeled with a detectable substance and an unlabeled antibody that specifically binds the desired protein (such as TPX2, KIF11, ZWILCH, MYC, DEPDC1, CDCA3, HMGB2, or CDC20, or one of the genes disclosed in Table 1). In this assay, the biological sample (such as a tissue biopsy, cells isolated from a tissue biopsy, blood, or urine), the labeled standards, and the antibody that specifically binds the desired protein are combined and the amount of labeled standard bound to the unlabeled antibody is determined. The amount of protein in the biological sample is inversely proportional to the amount of labeled standard bound to the antibody that specifically binds the protein of interest.

### V. Arrays

In particular embodiments provided herein, arrays are used to evaluate gene expression, for example to prognose a patient with cancer (for example, prostate cancer). When describing an array that consists essentially of probes or primers specific for one or more of the genes listed in Table 1, such an array includes probes or primers specific for these genes, and can further include control probes (for example to confirm the incubation conditions are sufficient). In some examples, the array may include or consist essentially of one or more (such as 1, 2, 3, 4, 5, 6, 7, or 8, for instance) probes or primers specific for one or more of TPX2, KIF11, ZWILCH, MYC, DEPDC1, CDCA3, HMGB2, and CDC20, and can further include one or more control probes. In other examples, the array may include or consist essentially of one or more probes or primers specific for one or more of the genes disclosed in Table 1, and can further include one or more control probes. Exemplary control probes include GAPDH, actin, and 18S RNA. In one example, an array is a multi-well plate (e.g., 96 or 384 well plate).

In one example, the array includes, consists essentially of, or consists of probes or primers (such as an oligonucleotide or antibody) that can recognize TPX2, KIF11, ZWILCH, MYC, DEPDC1, CDCA3, HMGB2, and CDC20. The probes or primers can further include one or more detectable labels, to permit detection of specific binding between the probe and target sequence (such as one of the genes disclosed herein).

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

A wide variety of array formats can be employed in accordance with the present disclosure. One example includes a linear array of oligonucleotide bands, peptides, or antibodies, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence.

The array formats of the present disclosure can be included in a variety of different types of formats. A “format” includes any format to which the solid support can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotide and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as PCT applications WO 85/01051 and WO 89/10977, or U.S. Pat. No. 5,554,501).

A suitable array can be produced to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells.

The oligonucleotides can be bound to the polypropylene support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for affixing to the solid support. In general, the internal complementarity of an oligonucleotide probe in the region of the 3′ end and the 5′ end determines binding to the support.

In particular examples, oligonucleotide probes on the array include one or more labels, that permit detection of oligonucleotide probe:target sequence hybridization complexes.

### VI. Diagnostic Kits

The methods described herein may be performed, for example, by utilizing diagnostic kits comprising at least one specific nucleic acid probe, which may be conveniently used, such as in clinical settings, to provide a prognosis for subjects with prostate cancer. Such kits may be provided in the form of a package, box, bag, or other container enclosing one or more components that may be used in determining the expression of TPX2, KIF11, ZWILCH, MYC, DEPDC1, CDCA3, HMGB2, and/or CDC20. Such kits may also contain labeling reagents, enzymes including PCR amplification reagents such as Taq or Pfu; reverse transcriptase and additional buffers and solutions that facilitate the performance of the method.

A diagnostic kit may contain reagents, such as antibodies, that specifically bind proteins. Such kits will contain one or more specific antibodies, buffers, and other reagents configured to detect binding of the antibody to the specific epitope. One or more of the antibodies may be labeled with a fluorescent, enzymatic, magnetic, metallic, chemical, or other label that signifies and/or locates the presence of specifically bound antibody. The kit may also contain one or more secondary antibodies that specifically recognize epitopes on other antibodies. These secondary antibodies may also be labeled. The concept of a secondary antibody also encompasses non-antibody ligands that specifically bind an epitope or label of another antibody. For example, streptavidin or avidin may bind to biotin conjugated to another antibody. Such a kit may also contain enzymatic substrates that change color or some other property in the presence of an enzyme that is conjugated to one or more antibodies included in the kit.

Kits may be provided as a reagent bound to a substrate material. For example, the kit may comprise an antibody or other protein reagent bound to a polystyrene plate. Alternatively, the kit may comprise a nucleic acid such as an oligonucleotide, bound to a substrate, wherein a substrate may be any solid or semi solid material onto which a nucleic acid, such as an oligonucleotide may be affixed, attached or printed, either singly or in a microarray format.

A diagnostic kit may also contain an indication of the threshold level of expression of TPX2, KIF11, ZWILCH, MYC, DEPDC1, CDCA3, HMGB2, and/or CDC20 that will signify that the subject has a poor prognosis in prostate cancer. An indication may be any communication of the threshold level of expression. The indication may further indicate that expression of TPX2, KIF11, ZWILCH, MYC, DEPDC1, CDCA3, HMGB2, and/or CDC20 above the threshold level of expression will signify that the subject has a poor prognosis. The indication of the threshold level may be provided in multiple stages such in a system that the subject has a high, medium or low risk of having a poor prognosis. The indication may comprise any number of stages. The indication may indicate the threshold of expression numerically, as in an optical density of an ELISA assay, a protein concentration (such as ng/ml), a percentage of cells expressing CCR6, or in fold-expression relative to a positive control, negative control, or housekeeping gene. The indication may be a positive or negative control that intended to be matched to the sample by eye or through an instrument. The indication may be a size marker to be compared to the sample through gel electrophoresis.

The indication may be communicated through any tangible medium of expression. It may be printed the packaging material, a separate piece of paper, or any other substrate and provided with the kit, provided separately from the kit, posted on the Internet, written into a software package. The indication may comprise an image such as a FACS image, a photograph or a photomicrograph, or any copy or other reproduction of these, particularly when TPX2, KIF11, ZWILCH, MYC, DEPDC1, CDCA3, HMGB2, and/or CDC20 expression is determined through the use of in situ hybridization, FACS analysis, or immunohistochemistry.

The diagnostic procedures can be performed “in situ” directly upon blood smears (fixed and/or frozen), or on tissue biopsies, such that no nucleic acid purification is necessary. DNA or RNA from a sample can be isolated using procedures which are well known to those in the art.

Nucleic acid reagents that are specific to the nucleic acid of interest, namely the nucleic acids encoding TPX2, KIF11, ZWILCH, MYC, DEPDC1, CDCA3, HMGB2, and/or CDC20, can be readily generated given the sequences of these genes for use as probes and/or primers for such in situ procedures (see, for example, Nuovo, G. J., 1992, PCR in situ hybridization: protocols and applications, Raven Press, NY).

### EXAMPLES

The following examples are illustrative of disclosed methods. In light of this disclosure, those of skill in the art will recognize that variations of these examples and other examples of the disclosed method would be possible without undue experimentation.

### Example 1

### Identification of Genes Involved in Androgen-Independent Prostate Cancer Cell Growth

Published data from 1) androgen receptor ChIP (chromatin immunoprecipitation)-Chip micro array data from castration-sensitive prostate cancer cell line LNCaP and its castration-resistant prostate cancer derivative call line (Abl) grown in androgen-free serum but stimulated with the synthetic androgen DHT (dihydrotestosterone); 2) gene expression profiles after RNAi-mediated suppression of the androgen receptor or a non-targeted control in LNCaP and Abl cells grown in androgen-free serum; and 3) gene expression profiles after the addition of DHT or vehicle to LNCaP or Abl cells grown in androgen-free serum (Wang et al., Cell 138:245-256. 20 2009) were analyzed.

A number of genes exhibited differential expression upon RNAi-mediated suppression of androgen receptor. Some of the differential expression occurred in one of the LNCaP or Abl lines but not the other. However, most of the genes that exhibited differential expression did so in both lines.

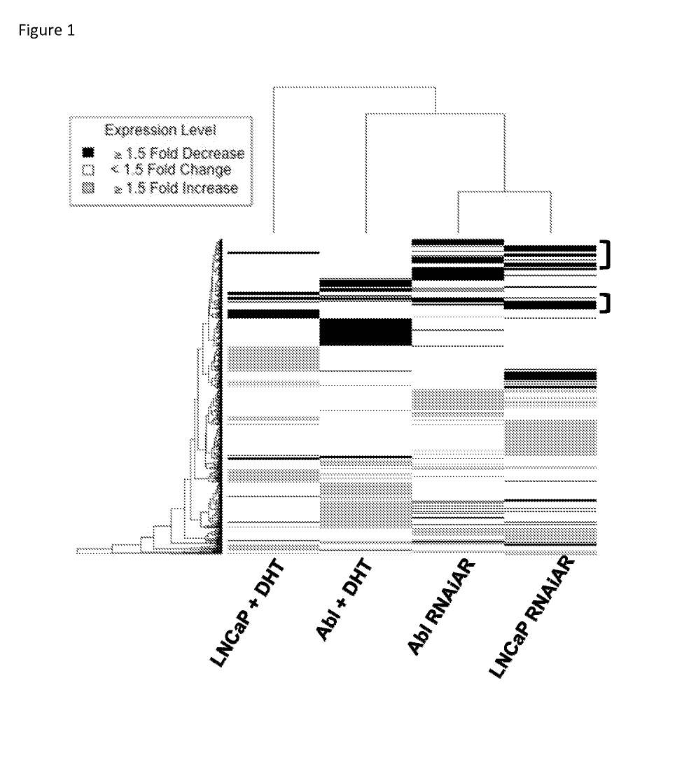

A minority of the genes known to be controlled by the androgen receptor exhibited lower expression with RNAi suppression of AR. Some of these same genes exhibited higher expression with the addition of androgens (FIG. 1; lanes 3 and 4 vs. lanes 1 and 2). Furthermore, AR was bound to these androgen-independent genes in the absence of androgens in ChIP assays, and adding androgens to LNCaP or Abl cells did not increase AR binding to these genes. This demonstrates that androgen-independent AR signaling is operational even in castration sensitive prostate cancer cells, and that these pathways are also relevant to castration resistant prostate cancer cells.

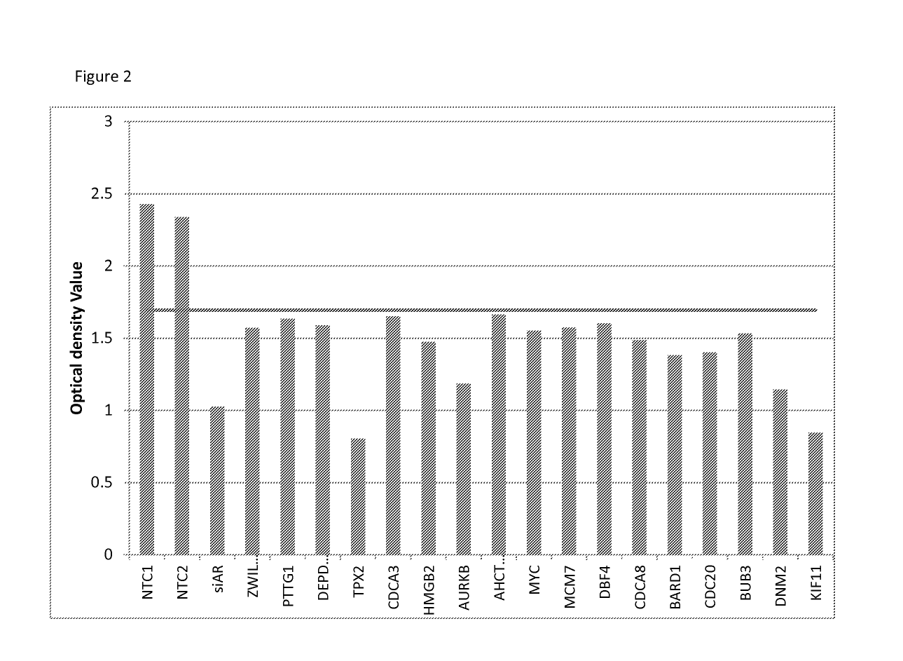

The expression of each of the androgen-independent AR target genes identified from the analysis in FIG. 1 was suppressed in order to identify genes that promote prostate cancer growth. This was accomplished using RAPID (RNAi-assisted protein target identification), a high-throughput, 96-well plate RNAi assay (Tyner et al., Proc. Natl. Acad. Sci. USA 5, 8695-8700 (2009), incorporated by reference herein.) Three different siRNAs per candidate androgen-independent AR target gene of interest or non-target control (NTC) siRNAs were introduced into LNCaP cells grown in androgen-free serum. Cell viability was quantified using the CellTiter 96® AQueous One Solution cell proliferation assay (Promega; Madison, Wis.). Results from a representative plate are shown in FIG. 2.

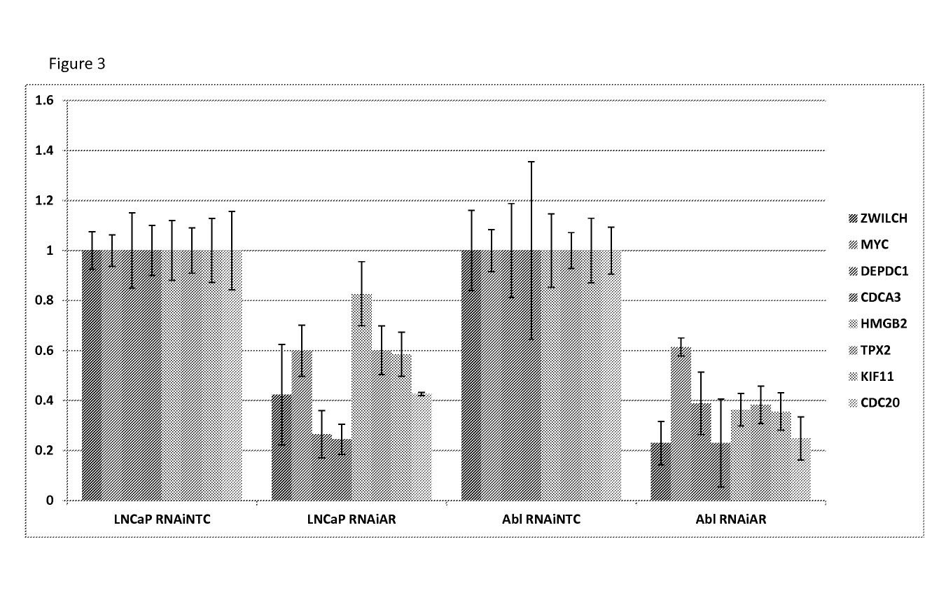

Twenty genes met the criteria of having at least two of the three siRNAs used causing a disruption in cell growth valued at more than one standard deviation below the median cell viability for each plate. These genes are listed in Table 1. Of those, RNAi suppression of ten genes (DEPDC1, TPX2, AURKB, MYC, MCM7, DBF4, BARD 1, CDC20, DNM2, and KIF11) also disrupted growth of castration resistant prostate cancer Abl cells. Those results are shown in FIG. 2. QRTPCR confirmed that RNAi-mediated suppression of AR in both LNCaP and CRPC Abl cells reduced expression of all of these genes. The data are summarized in FIG. 3.

TABLE 1
siRNA that silence growth in LNCaP cells.

| Gene Symbol | Gene Name | SEQ ID NO: |
|---|---|---|
| ZWILCH | Zwilch, kinetochore | SEQ ID NO: 1 |
| | associated homolog | |
| PTTG1 | Pituitary tumor-transforming 1 | SEQ ID NO: 2 |
| DEPDC1 | DEP domain containing 1 | SEQ ID NO: 3 |
| TPX2 | Tpx2, microtubule | SEQ ID NO: 4 |
| | associated homolog | |
| CDCA3 | Cell division cycle associated 3 | SEQ ID NO: 5 |
| BCCIP | BRCA2 and CDKN1 | SEQ ID NO: 6 |
| | interacting protein | |
| HMGB2 | High-mobility group box 2 | SEQ ID NO: 7 |
| AURKB | Aurora kinase B | SEQ ID NO: 8 |
| KPNA2 | Karyopherin alpha 2 (RAG | SEQ ID NO: 9 |
| | cohort 1, importin alpha 1) | |
| AHCTF1 | AT hook containing | SEQ ID NO: 10 |
| | transcription factor 1 | |
| MYC | v-myc myelocytomatosis | SEQ ID NO: 11 |
| | viral oncogene homolog | |
| MCM7 | Minichromosome maintenance | SEQ ID NO: 12 |
| | complex component 7 | |
| DBF4 | DBF4 homolog | SEQ ID NO: 13 |
| CDCA8 | Cell division cycle associated 8 | SEQ ID NO: 14 |
| BARD1 | BRCA1 associated RING | SEQ ID NO: 15 |
| | domain 1 | |
| SGOL2 | Shugoshin-like | SEQ ID NO: 16 |
| CDC20 | Cell division cycle 20 homolog | SEQ ID NO: 17 |
| BUB3 | Budding uninhibited by | SEQ ID NO: 18 |
| | benzimidazoles 3 | |
| DNM2 | Dynamin 2 | SEQ ID NO: 19 |
| KIF11 | Kinesin family member 11 | SEQ ID NO: 20 |

### Example 2

### Prognostic Impact of Androgen-Independent AR Target Genes

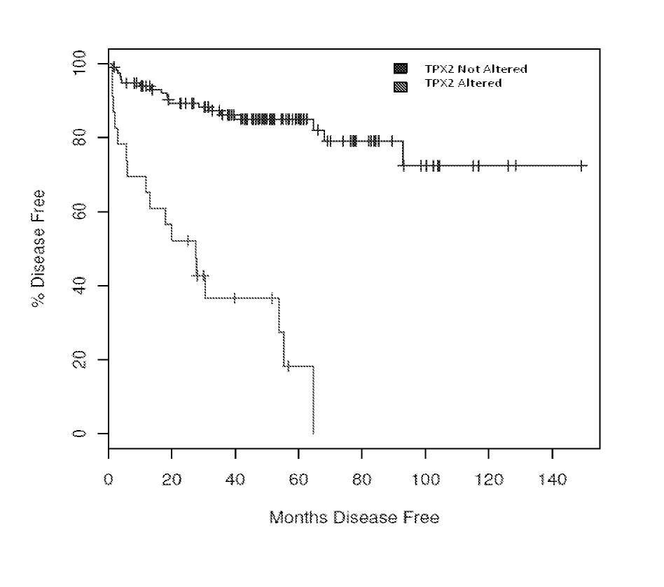

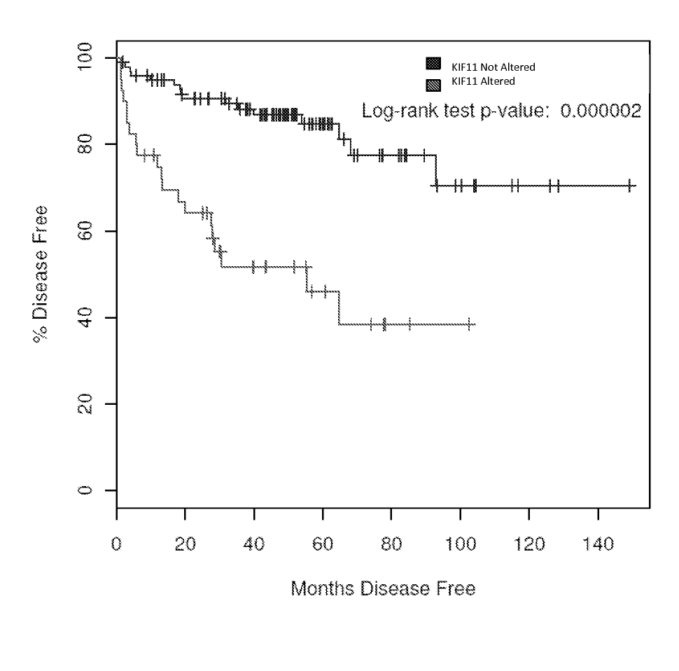

The expression levels of each of TPX2, KIF11, ZWILCH, MYC, DEPDC1, CDCA3, HMGB2, CDC20, AURKB, MCM7, DBF4, BARD1, CDC20, and DNM2 in prostate tumors at the time of diagnosis was analyzed in a published gene expression profile from prostate cancer samples (Taylor et al., Cancer Cell 18:11-22, 2010; cbioportal.org/cgx/index.do, incorporated by reference herein) using outlier analysis. Tumors with altered TPX2 or KIF11 are the tumors with the highest decile of expression of TPX2 (FIG. 4A) or KIF11 (FIG. 4B) in the dataset in the Taylor et al reference above. Subjects with a tumor with altered expression of TPX2 or KIF11 had a shorter relapse-free survival than patients without altered expression.

Expression of TPX2 in the tumor over the threshold indicated a 100% chance that a patient would relapse within at least 70 months. Expression of KIF11 in the tumor over the threshold indicated a 60% chance that a patient would relapse within 120 months.

One way of selecting a threshold level of expression of, for example, TPX2 would be to select tumor samples of at least 50, at least 75, at least 100, at least 150, at least 200, or more than 200 patients with prostate cancer, quantifying the expression of TPX2 mRNA, selecting the top 10% of samples with regard to mRNA expression of TPX2, and setting the threshold level of expression at the lowest level of expression of group consisting of the top 10% of samples in terms of TPX2 expression.

This example would work for any method of quantifying the expression of TPX2 mRNA, including any such method disclosed herein.

### Example 3

### Prognosis of a Subject with Prostate Cancer

This example describes particular representative methods that can be used to prognose a subject diagnosed with prostate cancer. However, one skilled in the art will appreciate that methods that deviate from these specific methods can also be used to successfully provide the prognosis of a subject with prostate cancer, based on the teachings provided herein.

A tumor sample is obtained from the subject. Approximately 1-100 μg of tissue is obtained for each sample type, for example using a fine needle aspirate. RNA and/or protein is isolated from the tumor sample using routine methods (for example using a commercial kit).

Prognosis of the prostate tumor is determined by detecting expression levels of one or more of TPX2, KIF11, ZWILCH, MYC, DEPDC1, CDCA3, HMGB2, and CDC20 in a tumor sample obtained from a subject by microarray analysis or real-time quantitative PCR. The relative expression level of one or more of TPX2, KIF11, ZWILCH, MYC, DEPDC1, CDCA3, HMGB2, and CDC20 in the tumor sample is compared to a threshold level of expression. One type of threshold level of expression may be expression in a control, such as RNA isolated from adjacent non-tumor tissue from the subject). In other cases, the threshold level of expression is a reference value, such as the relative amount of such molecules present in non-tumor samples obtained from a group of healthy subjects or cancer subjects. Preferably the threshold level of expression maximizes the sensitivity and selectivity of the test in determining prognosis.

The relative expression of one or more of TPX2, KIF11, ZWILCH, MYC, DEPDC1, CDCA3, HMGB2, and CDC20 is determined at the protein level by methods known to those of ordinary skill in the art, such as protein microarray, Western blot, or immunoassay techniques. Total protein is isolated from the tumor sample and compared to a control (e.g., protein isolated from adjacent non-tumor tissue from the subject or a reference value) using any suitable technique.

Expression of one or more of, or all of TPX2, KIF11, ZWILCH, MYC, DEPDC1, CDCA3, HMGB2, and CDC20 RNA or protein in the tumor sample over the threshold level of expression, about 1.5 fold, about 2-fold, about 2.5-fold, about 3-fold, about 4-fold, about 5-fold, about 7-fold or about 10-fold) indicates a poor prognosis, such as resistance to or risk of resistance to a therapy (such as ADT,) or likelihood to relapse or develop metastases.

The results of the test are provided to a user (such as a clinician or other health care worker, laboratory personnel, or patient) in a perceivable output that provides information about the results of the test. In some examples, the output can be a paper output (for example, a written or printed output), a display on a screen, a graphical output (for example, a graph, chart, or other diagram), or an audible output. In other examples, the output is a numerical value, such as an amount of expression of one or more genes in the sample or a relative amount of one or more genes in the sample as compared to a control. In a particular example, the output (such as a graphical output) shows or provides the threshold level of expression that indicates poor prognosis such that if the value or level of expression of one or more genes in the sample is above the threshold level of expression and good prognosis if the value or level of expression of one or more genes in the sample is below the threshold level of expression. In some examples, the output is communicated to the user, for example by providing an output via physical, audible, or electronic communication (for example by mail, telephone, facsimile transmission, email, or communication to an electronic medical record).

The output can provide quantitative information (for example, an amount of gene expression or gene expression relative to an internal control, external control, or threshold level of expression) or can provide qualitative information (for example, a prognosis). In additional examples, the output can provide qualitative information regarding the relative amount of gene expression in the sample, such as identifying presence of an increase in one or more protein relative to a control.

In some examples, the output is accompanied by guidelines for interpreting the data, for example, numerical or other limits that indicate a prognosis. The indicia in the output can, for example, include normal or abnormal ranges or a cutoff, which the recipient of the output may then use to interpret the results, for example, to arrive at a prognosis, or treatment plan. In other examples, the output can provide a recommended therapeutic regimen (for example, based on the amount of gene expression or the amount of increase of gene expression relative to a control), such as selection of one or more hormone therapies, radiation therapy, chemotherapy, or a combination of two or more thereof.

In view of the many possible embodiments to which the principles of the disclosure may be applied, it should be recognized that the illustrated embodiments are only examples and should not be taken as limiting the scope of the invention. Rather, the scope of the invention is defined by the following claims.

1. A method of predicting whether or not a subject with prostate cancer will relapse, the method comprising:
obtaining a biological sample from the subject, wherein the biological sample comprises RNA from prostate cancer cells;
contacting the biological sample with at least one oligonucleotide complementary to part of SEQ ID NO: 4 or SEQ ID NO: 20;
performing nucleic acid amplification on the biological sample;
determining an expression level of SEQ ID NO: 4 or SEQ ID NO: 20 from the nucleic acid amplification; and
comparing the expression level of SEQ ID NO: 4 or SEQ ID NO: 20 with a threshold level of expression;
wherein an expression level of SEQ ID NO: 4 or SEQ ID NO: 20 that exceeds the threshold level of expression indicates that the subject is predicted to relapse.

2. The method of claim 1 wherein performing nucleic acid amplification on the biological sample comprises performing reverse transcription polymerase chain reaction.

3. (canceled)

4. The method of claim 1, wherein the threshold level of expression comprises the expression level of a positive control.

5-11. (canceled)

12. The method of claim 1 further comprising comparing the level of expression of SEQ ID NO: 4 or SEQ ID NO: 20 in the sample with the expression level of SEQ ID NO: 4 or SEQ ID NO: 20 in non-cancerous cells from the same subject.

13. (canceled)

14. The method of claim 1 wherein the relapse is predicted to occur in less than 70 months following treatment.

15. The method of claim 14, wherein the relapse is predicted to occur between 1 and 30 months following treatment.

16. The method of claim 1 wherein the subject is human.

17. A kit used in performing the method of claim 1, the kit comprising:
at least one oligonucleotide that specifically binds to a nucleic acid of SEQ ID NO: 4 or SEQ ID NO: 20; and
an indication of a threshold level of expression of SEQ ID NO: 4 or SEQ ID NO: 20, wherein expression of SEQ ID NO: 4 or SEQ ID NO: 20 that exceeds the threshold level of expression signifies that the subject is predicted to relapse.

18-19. (canceled)

20. The kit of claim 17, wherein the at least one oligonucleotide is a PCR primer.

21. The kit of claim 20, further comprising at least one oligonucleotide probe configured for use in quantitative reverse transcription polymerase chain reaction.

22. The kit of claim 21 wherein the at least one oligonucleotide probe comprises a label.

23-28. (canceled)

29. The kit of claim 17 wherein the indication comprises a control configured to provide a result similar to that of the threshold level of expression.

30. The kit of claim 17, comprising:
at least one oligonucleotide that specifically binds to a nucleic acid of SEQ ID NO: 4 and at least one oligonucleotide that specifically binds to a nucleic acid of SEQ ID NO: 20; and
an indication of the threshold level of expression of SEQ ID NO: 4 and an indication of the threshold expression level of SEQ ID NO: 20.

31. The kit of claim 21, wherein the at least one probe comprises at least one probe that specifically binds to a nucleic acid of SEQ ID NO: 4, at least one probe that specifically binds to a nucleic acid of SEQ ID NO: 20, or a combination thereof.

32. The method of claim 1, wherein contacting the biological sample with at least one oligonucleotide complementary to part of SEQ ID NO: 4 or SEQ ID NO: 20 comprises contacting the biological sample with at least one oligonucleotide complementary to part of SEQ ID NO: 4 and at least one oligonucleotide complementary to part of SEQ ID NO: 20.


SEQUENCE LISTING (21 sequences)

SEQ ID NO: 1 - DNA, 3194 bp, Homo sapiens
agtcgaggtatcttctccccaaccactgctcttattttaattattgcagacggaagttgaagactattgacatagtaaatagctctgggtggcttgaaacgaaagtttaactttgcggacaaacaggacttattgtagggggtggtcaaaatagtcccggcggggcggggccatgacccctgacgtcgccggtccggcgcgcagttcagtttggcggttccggtaccgctctcacattggggcgggatgtgggagcggctgaactgcgcagcagaggacttttattctcgtctccttcagaaatttaatgaagaaaagaaaggaatccgtaaagacccatttctctatgaggctgatgtccaagtgcagttgatcagcaaaggccaaccaaaccctttgaaaaatattctaaatgaaaatgacatagtattcatagtggaaaaagtgcctttagaaaaggaagaaacaagtcatattgaagaacttcaatctgaagaaactgccatatctgatttctctactggcgaaaatgttggaccacttgctttaccagttgggaaggcaaggcagttaattggactttacaccatggctcacaatcctaatatgacccatttgaagattaatctgccagttactgcccttcctcccctttgggtaagatgtgacagttcagatcctgaaggtacttgttggctaggagctgagcttatcacaacaaacaacagcattacaggaattgtcttatatgtggtcagttgtaaagctgataaaaattattctgtaaatcttgaaaacctaaaaaatttacacaagaaaagacatcacttgtctactgtaacatccaaaggctttgcccagtatgagctctttaagtcctctgccttggatgatacaatcacagcatcacaaactgcgatcgctttggatatttcctggagtcctgtggatgagattcttcaaatccctccactctcttcaactgcaactctgaatattaaagtggaatcaggagagcccagaggtcctttgaatcatctctacagagaactgaaatttcttcttgttttggctgatggtttgaggactggtgtcactgaatggctcgagcccctggaagcaaaatctgctgttgaacttgttcaggaatttctgaatgacttaaataagctggatggatttggtgattctacaaaaaaagacactgaggttgagaccttgaagcatgacactgctgcagtcgatcgttccgtcaagcgtcttttcaaagttcggagtgatcttgattttgctgagcaactgtggtgcaaaatgagcagtagtgtgatttcataccaagacttggtgaagtgtttcacattgatcatccagagtctacaacgtggtgatatacagccatggctccatagtggaagtaacagtttactaagtaagctcattcatcagtcttatcatggaaccatggacacagtttctctcagtgggactattccagttcaaatgcttttggaaattggtttggacaaactaaagaaagattatatcagttttttcataggtcaggaacttgcatctttgaatcatttggaatacttcattgctccatcagtagatatacaagaacaggtttatcgtgtccaaaaactccaccatattctagaaatattagtcagttgcatgcctttcattaaatctcaacatgaactcctcttttctttaacacagatctgcataaagtattacaaacaaaatcctcttgatgagcaacacatttttcagctgccagtcagaccaactgctgtaaagaacttatatcaaagtgagaagccacagaaatggagagtggaaatatatagtggtcaaaagaagattaagacagtttggcaactgagtgacagctcacccatagaccatctgaattttcacaaacctgatttttcggaattaacactaaacggtagcctggaagaaaggatattctttactaacatggttacctgcagccaggtgcatttcaagtgaagtgtgctgatgaagtcctctataagcacaagccaaaaagagaaagagaaaaaaaggtaattattgtagaacctgaaaacagcaatgtatggaaaccctcaaagcagaaaagggaggaagatcctgaagattctcttatgaagctccaaaattgataatcctgtctcagctctgcctcctcaggaggagcattagtagaacagcagtgatgaggacacagagggagcagacagtgggtaccacgatctccgtaaccatttgcatgtgacttagcaagggctctgaaatgacaaagagaacgagcaccacaaatgagaacaggatcattttagtaaatacagctttatcccaaaagctttaactgtattgggaaaacttaaaaaatagcatcctcaaattttctgattcttatttgccatgaaatagaacttagtaaattaaatgttatttgaaaatgttataagagctttgtaaatatttcagaaaatatgggataaatgcctgaatttggttcttctacaggtgctataataaagtccatctctcaatacttatactttctaaattcatctcagaatattagcagccatattccacagttcctataatttttactgggggggatttgtgataggaaagtccttgggaaacatttccaatctttcaaaatattattgtgtatcttaagaagtataggaacttgtatgttgaaatgttgtatggtagttcttgtatagttaaataataatctttttaagagttaatgataagcatatgttatgtgcattattaataaaatagtggccacttaggtaatacccacttttatcttgtgtgctgggtactctggttactgagataaataaggcactggacatcctcacgtggagttcacaggctcatcagtgaattctgtaccacatttcaaccttgtttattttagtttaatggaatatacattcttagtattgcctgattatttaaatttgttgagggggattgcatgttgctttattggcctgtaaaaatagctagtttggtaagatttggtctcgcaccttccatctttgctaccacattaaagatgagcttgttaaaaaggaaagcatatttctctgattgcccttatggagaaataaagataaaattcaaagaaacaaaaaaaaaaaaaa

SEQ ID NO: 2 - DNA, 610 bp, Homo sapiens
atggctactctgatctatgttgataaggaaaatggagaaccaggcacccgtgtggttgctaaggatgggctgaagctggggtctggaccttcaatcaaagccttagatgggagatctcaagtttcaacaccacgttttggcaaaacgttcgatgccccaccagccttacctaaagctactagaaaggctttgggaactgtcaacagagctacagaaaagtctgtaaagaccaagggacccctcaaacaaaaacagccaagcttttctgccaaaaagatgactgagaagactgttaaagcaaaaagctctgttcctgcctcagatgatgcctatccagaaatagaaaaattctttcccttcaatcctctagactttgagagttttgacctgcctgaagagcaccagattgcgcacctccccttgagtggagtgcctctcatgatccttgacgaggagagagagcttgaaaagctgtttcagctgggccccccttcacctgtgaagatgccctctccaccatgggaatccaatctgttgcagtctccttcaagcattctgtcgaccctggatgttgaattgccacctgtttgctgtgacatagatatttaaa

SEQ ID NO: 3 - DNA, 4504 bp, Homo sapiens
tatgctattcaaatcggcggcggggccaacggttgtgccgagactcgccactgccgcggccgctgggcctgagtgtcgccttcgccgccatggacgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaagatcaagaaaatgcaattgataatagagaactaagccaggaagatgttgaagaagtttggagatatgttattctgatctacctgcaaaccattttaggtgtgccatccctagaagaagtcataaatccaaaacaagtaattccccaatatataatgtacaacatggccaatacaagtaaacgtggagtagttatactacaaaacaaatcagatgacctccctcactgggtattatctgccatgaagtgcctagcaaattggccaagaagcaatgatatgaataatccaacttatgttggatttgaacgagatgtattcagaacaatcgcagattattttctagatctccctgaacctctacttacttttgaatattacgaattatttgtaaacattttgggcttgctgcaacctcatttagagagggttgccatcgatgctctacagttatgttgtttgttacttcccccaccaaatcgtagaaagcttcaacttttaatgcgtatgatttcccgaatgagtcaaaatgttgatatgcccaaacttcatgatgcaatgggtacgaggtcactgatgatacataccttttctcgatgtgtgttatgctgtgctgaagaagtggatcttgatgagcttcttgctggaagattagtttctttcttaatggatcatcatcaggaaattcttcaagtaccctcttacttacagactgcagtggaaaaacatcttgactacttaaaaaagggacatattgaaaatcctggagatggactatttgctcctttgccaacttactcatactgtaagcagattagtgctcaggagtttgatgagcaaaaagtttctacctctcaagctgcaattgcagaacttttagaaaatattattaaaaacaggagtttacctctaaaggagaaaagaaaaaaactaaaacagtttcagaaggaatatcctttgatatatcagaaaagatttccaaccacggagagtgaagcagcactttttggtgacaaacctacaatcaagcaaccaatgctgattttaagaaaaccaaagttccgtagtctaagataactaactgaattaaaaattatgtaatacttgtggaactttgataaatgaagccatatctgagaatgtagctactcaaaaggaagtctgtcattaataaggtatttctaaataaacacattatgtaaggaagtgccaaaatagttatcaatgtgagactcttaggaaactaactagatctcaattgagagcacataacaatagatgataccaaatactttttgtttttaacacagctatccagtaaggctatcatgatgtgtgctaaaattttatttacttgaattttgaaaactgagctgtgttagggattaaactataattctgttcttaaaagaaaatttatctgcaaatgtgcaagttctgagatattagctaatgaattagttgtttggggttacttctttgtttctaagtataagaatgtgaagaatatttgaaaactcaatgaaataattctcagctgccaaatgttgcactcttttatatattctttttccacttttgatctatttatatatatgtatgtgtttttaaaatatgtgtatattttatcagatttggttttgccttaaatattatccccaattgcttcagtcattcatttgttcagtatatatattttgaattctagttttcataatctattagaagatggggatataaaagaagtataaggcaatcatatattcattcaaaagatatttatttagcaactgctatgtgcctttcgttgttccagatatgcagagacaatgataaataaaacatataatctcttccataaggtatttattttttaatcaagggagatacacctatcagatgtttaaaataacaacactacccactgaaatcagggcatatagaatcattcagctaaagagtgacttctatgatgatggaacaggtctctaagctagtggttttcaaactggtacacattagactcacccgaggaattttaaaacagcctatatgcccagggcctaacttacactaattaaatctgaattttggggatgttgtatagggattagtattttttttaatctaggtgattccaatattcagccaactgtgagaatcaatggcctaaatgctttttataaacatttttataagtgtcaagataatggcacattgactttattttttcattggaagaaaatgcctgccaagtataaatgactctcatcttaaaacaaggttcttcaggtttctgcttgattgacttggtacaaacttgaagcaagttgccttctaatttttactccaagattgtttcatatctattccttaagtgtaaagaaatatataatgcatggtttgtaataaaatcttaatgtttaatgactgttctcatttctcaatgtaatttcatactgtttctctataaaatgatagtattccatttaacattactgatttttattaaaaacctggacagaaaattataaattataaatatgactttatcctggctataaaattattgaaccaaaatgaattctttctaaggcatttgaatactaaaacgtttattgtttatagatatgtaaaatgtggattatgttgcaaattgagattaaaattatttggggttttgtaacaatataattttgcttttgtattatagacaaatatataaataataaaggcaggcaactttcatttgcactaatgtacatgcaattgagattacaaaatacatggtacaatgctttaataacaaactctgccagtcaggtttgaatcctactgtgctattaactagctagtaaactcagacaagttacttaacttctctaagccccagttttgttatctataaaatgaatattataatagtacctctttttaggattgcgaggattaagcaggataatgcatgtaaagtgttagcacagtgtctcacatagaataagcactctataaatattttactagaatcacctaggattatagcactagaagagatcttagcaaaaatgtggtcctttctgttgctttggacagacatgaaccaaaacaaaattacggacaattgatgagccttattaactatcttttcattatgagacaaaggttctgattatgcctactggttgaaattttttaatctagtcaagaaggaaaatttgatgaggaaggaaggaatggatatcttcagaagggcttcgcctaagctggaacatggatagattccattctaacataaagatctttaagttcaaatatagatgagttgactggtagatttggtggtagttgctttctcgggatataagaagcaaaatcaactgctacaagtaaagaggggatggggaaggtgttgcacatttaaagagagaaagtgtgaaaaagcctaattgtgggaatgcacaggtttcaccagatcagatgatgtctggttattctgtaaattatagttcttatcccagaaattactgcctccaccatccctaatatcttctaattggtatcatataatgacccactcttcttatgttatccaaacagttatgtggcatttagtaatggaatgtacatggaatttcccactgacttacctttctgtccttgggaagcttaaactctgaatcttctcatctgtaaaatgtgaattaaagtatctacctaactgagttgtgattgtagtgaaagaaaggcaatatatttaaatcttgaatttagcaagcccacgcttgatttttatgtcctttcctcttgccttgtattgagtttaagatctctactgattaaaactcttttgctatcaaaaaaaaaaaaaaaaaaaaaaaaa

SEQ ID NO: 4 - DNA, 3685 bp, Homo sapiens
agtggactcacgcaggcgcaggagactacacttcccaggaactccgggccgcgttgttcgctggtacctccttctgacttccggtattgctgcggtctgtagggccaatcgggagcctggaattgctttcccggcgctctgattggtgcattcgactaggctgcctgggttcaaaatttcaacgatactgaatgagtcccgcggcgggttggctcgcgcttcgttgtcagatctgaggcgaggctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaagaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctcaaaaaaaaaaaaaaaaaaaaaaa

SEQ ID NO: 5 - DNA, 1170 bp, Homo sapiens
aagtttgaaactggtaacttcgggagttgagccacgagctgttgtgcatccagaggtggaattggggcccggcattccctcctcgtcccgggctggcccttgcccccaccctgcaactcctggttgagatgggctcagccaagagcgtcccagtcacaccagcgcggcctccgccgcacaacaagcatctggctcgagtggcggacccccgttcacctagtgctggcatcctgcgcactcccatccaggtggagagctctccacagccaggcctaccagcaggggagcaactggagggtcttaaacatgcccaggactcagatccccgctctcctactcttggtattgcacggacacctatgaagaccagcagtggagaccccccaagcccactggtgaaacagctgagtgaagtatttgaaactgaagactctaaatcaaatcttcccccagagcctgttctgcccccagaggcacctttatcttctgaattggacttgcctctgggtacccagttatctgttgaggaacagatgccaccttggaaccagactgagttcccctccaaacaggtgttttccaaggaggaagcaagacagcccacagaaacccctgtggccagccagagctccgacaagccctcaagggaccctgagactcccagatcttcaggttctatgcgcaatagatggaaaccaaacagcagcaaggtactagggagatcccccctcaccatcctgcaggatgacaactcccctggcaccctgacactacgacagggtaagcggccttcacccctaagtgaaaatgttagtgaactaaaggaaggagccattcttggaactggacgacttctgaaaactggaggacgagcatgggagcaaggccaggaccatgacaaggaaaatcagcactttcccttggtggagagctaggccctgcatggccccagcaatgcagtcacccagggcctggtgatatctgtgtcctctcaccccttctttcccagggatactgaggaatggcttgttttcttagactcctcctcagctaccaaactgggactcacagctttattgggctttctttgtgtcttgtgtgtttcttttatattaaaggaagtaattttaaatgttactttaaaaaggtatatgtaaaccttgcaccgag

SEQ ID NO: 6 - DNA, 1272 bp, Homo sapiens
gggggtgagcggcaacatggcgtccaggtctaagcggcgtgccgtggaaagtggggttccgcagccgccggatcccccagtccagcgcgacgaggaagaggaaaaagaagtcgaaaatgaggatgaagacgatgatgacagtgacaaggaaaaggatgaagaggacgaggtcattgacgaggaagtgaatattgaatttgaagcttattccctatcagataatgattatgacggaattaagaaattactgcagcagctttttctaaaggctcctgtgaacactgcagaactaacagatctcttaattcaacagaaccatattgggagtgtgattaagcaaacggatgtttcagaagacagcaatgatgatatggatgaagatgaggtttttggtttcataagccttttaaatttaactgaaagaaagggtacccagtgtgttgaacaaattcaagagttggttctacgcttctgtgagaagaactgtgaaaagagcatggttgaacagctggacaagtttttaaatgacaccaccaagcctgtgggccttctcctaagtgaaagattcattaatgtccctccacagatcgctctgcccatgtaccagcagcttcagaaagaactggcgggggcacacagaaccaataagccatgtgggaagtgctacttttaccttctgattagtaagacatttgtggaagcagaaaaaaacaattccaaaaagaaacctagcaacaaaaagaaagctgcgttaatgtttgcaaatgcagaggaagaatttttctatgagaaggcaattctcaagttcaactactcagtgcaggaggagagcgacacttgtctgggaggcaaatggtcttttgatgacgtaccaatgacgcccttgcgaactgtgatgttaattccaggcgacaagatgaacgaaatcatggataaactgaaagaatatctatctgtctaacccatttccaatggacagtgatgggcttgtttttgtaaaattaccagaaaactcagtggagatttactgaaaaactcagactttattcagattaagttcctctacaaaaagtagggttctgtcccatgtgtctctgacacatttacaaaataccagttttttaaaattttggtcaaattatgagtggttgatttaaaaacttttccaagaagaagaaaagcatggagtcgtaatttaaagaactcaataaaaacttctattttttattttaaaataataccaaaaaaaaaaaaaaaaaaaaaaaaaaaaaa

SEQ ID NO: 7 - DNA, 1527 bp, Homo sapiens
ggggatgtggcccgtggcctagctcgtcaagttgccgtggcgcggagaactctgcaaaacaagaggctgaggattgcgttagagataaaccagttcacgccggagccccgtgagggaagcgtctccgttgggtccggccgctctgcgggactctgaggaaaagctcgcaccaggtggacgcggatctgtcaacatgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataaatggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatattatttctattgtctttgtcttaatatgctaagttaattttcactttaaaaaagccatttgaagaccagagctatgttgatttttttcggtatttctgcctagtagttcttagacacagttgacctagtaaaatgtttgagaattaaaaccaaacatgctcatatttgcaaaatgttctttaaaagttacatgttgaactcagtgaactttataagaatttatgcagttttacagaacgttaagttttgtacttgacgtttctgtttattagctaaattgttcctcaggtgtgtgtatatatatatacatatatatatatatatatat

SEQ ID NO: 8 - DNA, 1244 bp, Homo sapiens
gggagagtagcagtgccttggaccccagctctcctccccctttctctctaaggatggcccagaaggagaactcctacccctggccctacggccgacagacggctccatctggcctgagcaccctgccccagcgagtcctccggaaagagcctgtcaccccatctgcacttgtcctcatgagccgctccaatgtccagcccacagctgcccctggccagaaggtgatggagaatagcagtgggacacccgacatcttaacgcggcacttcacaattgatgactttgagattgggcgtcctctgggcaaaggcaagtttggaaacgtgtacttggctcgggagaagaaaagccatttcatcgtggcgctcaaggtcctcttcaagtcccagatagagaaggagggcgtggagcatcagctgcgcagagagatcgaaatccaggcccacctgcaccatcccaacatcctgcgtctctacaactatttttatgaccggaggaggatctacttgattctagagtatgccccccgcggggagctctacaaggagctgcagaagagctgcacatttgacgagcagcgaacagccacgatcatggaggagttggcagatgctctaatgtactgccatgggaagaaggtgattcacagagacataaagccagaaaatctgctcttagggctcaagggagagctgaagattgctgacttcggctggtctgtgcatgcgccctccctgaggaggaagacaatgtgtggcaccctggactacctgcccccagagatgattgaggggcgcatgcacaatgagaaggtggatctgtggtgcattggagtgctttgctatgagctgctggtggggaacccaccctttgagagtgcatcacacaacgagacctatcgccgcatcgtcaaggtggacctaaagttccccgcttctgtgcccacgggagcccaggacctcatctccaaactgctcaggcataacccctcggaacggctgcccctggcccaggtctcagcccacccttgggtccgggccaactctcggagggtgctgcctccctctgcccttcaatctgtcgcctgatggtccctgtcattcactcgggtgcgtgtgtttgtatgtctgtgtatgtataggggaaagaagggatccctaactgttcccttatctgttttctacctcctcctttgtttaataaaggctgaagctttttgtaaaaaaaaaaaaaaaaaaaaaaaa

SEQ ID NO: 9 - DNA, 1921 bp, Homo sapiens
gctgagtcgaggtggaccctttgaacgcagtcgccctacagccgctgattccccccgcatcgcctcccgtggaagcccaggcccgcttcgcagctttctccctttgtctcataaccatgtccaccaacgagaatgctaatacaccagctgcccgtcttcacagattcaagaacaagggaaaagacagtacagaaatgaggcgtcgcagaatagaggtcaatgtggagctgaggaaagctaagaaggatgaccagatgctgaagaggagaaatgtaagctcatttcctgatgatgctacttctccgctgcaggaaaaccgcaacaaccagggcactgtaaattggtctgttgatgacattgtcaaaggcataaatagcagcaatgtggaaaatcagctccaagctactcaagctgccaggaaactactttccagagaaaaacagccccccatagacaacataatccgggctggtttgattccgaaatttgtgtccttcttgggcagaactgattgtagtcccattcagtttgaatctgcttgggcactcactaacattgcttctgggacatcagaacaaaccaaggctgtggtagatggaggtgccatcccagcattcatttctctgttggcatctccccatgctcacatcagtgaacaagctgtctgggctctaggaaacattgcaggtgatggctcagtgttccgagacttggttattaagtacggtgcagttgacccactgttggctctccttgcagttcctgatatgtcatctttagcatgtggctacttacgtaatcttacctggacactttctaatctttgccgcaacaagaatcctgcacccccgatagatgctgttgagcagattcttcctaccttagttcggctcctgcatcatgatgatccagaagtgttagcagatacctgctgggctatttcctaccttactgatggtccaaatgaacgaattggcatggtggtgaaaacaggagttgtgccccaacttgtgaagcttctaggagcttctgaattgccaattgtgactcctgccctaagagccatagggaatattgtcactggtacagatgaacagactcaggttgtgattgatgcaggagcactcgccgtctttcccagcctgctcaccaaccccaaaactaacattcagaaggaagctacgtggacaatgtcaaacatcacagccggccgccaggaccagatacagcaagttgtgaatcatggattagtcccattccttgtcagtgttctctctaaggcagattttaagacacaaaaggaagctgtgtgggccgtgaccaactataccagtggtggaacagttgaacagattgtgtaccttgttcactgtggcataatagaaccgttgatgaacctcttaactgcaaaagataccaagattattctggttatcctggatgccatttcaaatatctttcaggctgctgagaaactaggtgaaactgagaaacttagtataatgattgaagaatgtggaggcttagacaaaattgaagctctacaaaaccatgaaaatgagtctgtgtataaggcttcgttaagcttaattgagaagtatttctctgtagaggaagaggaagatcaaaacgttgtaccagaaactacctctgaaggctacactttccaagttcaggatggggctcctgggacctttaacttttagatcatgtagctgagacataaatttgttgtgtactacgtttggtattttgtcttattgtttctctactaagaactctttcttaaatgtggtttgttactgtagcactttttacactgaaactatacttgaacagttccaactgtacatacatactgtatgaagcttgtcctctgactaggtttctaatttctatgtggaatttcctatcttgcagc

SEQ ID NO: 10 - DNA, 2412 bp, Homo sapiens
ggaactttcgatggtgatgagctctaagaaaaaacttacaaaaaagactgaaagtcaaagccaaaaacgttcattgcactcagtatcagaagaacgcacagatgaaatgacacataaagaaacaaatgagcaggaagaaagattgctcgccacagcttccttcactaaatcatcccgcagcagcaggactcggtctagcaaggccatcttgttgccggacctttctgaaccaaacaatgagcctttattttctccagcgtcagaagttccaaggaaagcaaaagctaaaaaaatagaggttcctgcacagctgaaagaattagtttcggatttatcttctcagtttgtcatctcacctcctgctttaaggagcagacaaaaaaacacatccaataagaacaagcttgaagatgaactgaaagatgatgcacaatcagtagaaactctgggagagccaaaagcgaaacgaatcaggacgtcaaaaacaaaacaagcaagcaaaaacacagaaaaagaaagtgcttggtcacctcctcccatagaaattcggctgatttcccccttggctagcccagctgacggagtcaagagcaaaccaagaaaaactacagaagtgacaggaacaggtcttggaaggaacagaaagaaactgtcttcctatccaaagcaaattttacgcagaaaaatgctgtaatttcttgggaagattttaatgtacacctatttgtaaagtcatcagaatagtgtggattattaaatatctagtttggaagaaaataatttatataaattattgtaaatttttatgtaaacagaaggtcttcaataagtaaagtaactccatatggagtgattgtttcagtccaggcaatttttctattttatattaagacttcatacatttatatatgtaaatatggcttattaatggaatgttaaataaaatgtatacttcacagtcgtttgtgtcttggatttttgaaagggaggggatatctgtttaaatagttttatatgctcattggtctcattttctctataattaaaatactagaccagtcttaaaatggggatgattgaagtattgatatttctttttacagttactattttataatttatgcactttgattctgtgattcagatttctaatcagaaaatgtatttttttgtttttggctgttactatgttaaaattgaattatgggcatgtcattttgccatctttgtagtttcacaaattttgtgtaatctacctcaaatgaataatccaagtattggttaactataatgttggcatctcttattcggcaagcttaaaggctctttaaagtcttaattagtcaaagactaatccaggttagattgaccggttcactgctcacttgcaaccttatcaaagggtttgacaaagggaaatgtaaaataaatctgtttatggatattgagtgcatcttgtatgtgcctaatattgataggatgagatgtctgaacaaatttttataatattgctgtgaaggagcttgctattgaaccacagaaatcccttaatattcaggttttaaaactggcaaattctcacaggacctcaggcacagattattgaggttgggagagagtgagtagatgtagaaaaggagaaaaacaacacacgccctgttctctacagtacaactgtgtgcaattaagcaatggtacttgatgtaggctctaacactcatcaataaataagtgttgtaaaataatttataacaggtaatcgatagtgtgtaatgaatggactattaataattgattatctagaaacgaactgctttcgtgggcttttaatattttaatgtgaagcatatgcagtgtgctttctgcatttatttttctaccaaataatacagataatgagaaattggtgaaaatgcctacgcaaagtgttgacagtgtgaaagcagtgcgagtgcggccttttagtcaggttagtgatggatgttacgctgccttgttgaaaatttcactgactttgattttattacttttttaatgatagttatcaaacttgtatttaagctgcttgtcatttatggaatattgaacttatttaaatgaacttgttaaatgaataaagagctaaacataattcagtaaacaattcctttgcgcaagtagcacaataaacatggatgcaacgtatgtcaagttaatacttttttaaaccaacgcaatttggtgaatatagatgtgtggtacctgtttttaataagtgtactttttttcccccctccgtgaatgtagatcataagcaaacaaattgcctgttctaaatgaactttacatatattttaaatgaatgtatgtacttacgtataaatgtctttatatagcttgaataaaaacactgctcattaaaaaaaaaaaaaa

SEQ ID NO: 11 - DNA, 2379 bp, Homo sapiens
gacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaaaaaaaaaaaaaaa

SEQ ID NO: 12 - DNA, 2412 bp, Homo sapiens
gtccaccgcgcggagattctcagcttccccaggagcaagacctctgagcccgccaagcgcggccgcacggccctcggcagcgatggcactgaaggactacgcgctagagaaggaaaaggttaagaagttcttacaagagttctaccaggatgatgaactcgggaagaagcagttcaagtatgggaaccagttggttcggctggctcatcgggaacaggtggctctgtatgtggacctggacgacgtagccgaggatgaccccgagttggtggactcaatttgtgagaatgccaggcgctacgcgaagctctttgctgatgccgtacaagagctgctgcctcagtacaaggagagggaagtggtaaataaagatgtcctggacgtttacattgagcatcggctaatgatggagcagcggagtcgggaccctgggatggtccgaagcccccagaaccagtaccctgctgaactcatgcgcagatttgagctgtattttcaaggccctagcagcaacaagcctcgtgtgatccgggaagtgcgggctgactctgtggggaagttggtaactgtgcgtggaatcgtcactcgtgtctctgaagtcaaacccaagatggtggtggccacttacacttgtgaccagtgtggggcagagacctaccagccgatccagtctcccactttcatgcctctgatcatgtgcccaagccaggagtgccaaaccaaccgctcaggagggcggctgtatctgcagacacggggctccagattcatcaaattccaggagatgaagatgcaagaacatagtgatcaggtgcctgtgggaaatatccctcgtagtatcacggtgctggtagaaggagagaacacaaggattgcccagcctggagaccacgtcagcgtcactggtattttcttgccaatcctgcgcactgggttccgacaggtggtacagggtttactctcagaaacctacctggaagcccatcggattgtgaagatgaacaagagtgaggatgatgagtctggggctggagagctcaccagggaggagctgaggcaaattgcagaggaggatttctacgaaaagctggcagcttcaatcgccccagaaatatacgggcatgaagatgtgaagaaggcactgctgctcctgctagtcgggggtgtggaccagtctcctcgaggcatgaaaatccggggcaacatcaacatctgtctgatgggggatcctggtgtggccaagtctcagctcctgtcatacattgatcgactggcgcctcgcagccagtacacaacaggccggggctcctcaggagtggggcttacggcagctgtgctgagagactccgtgagtggagaactgaccttagagggtggggccctggtgctggctgaccagggtgtgtgctgcattgatgagttcgacaagatggctgaggccgaccgcacagccatccacgaggtcatggagcagcagaccatctccattgccaaggccggcattctcaccacactcaatgcccgctgctccatcctggctgccgccaaccctgcctacgggcgctacaaccctcgccgcagcctggagcagaacatacagctacctgctgcactgctctcccggtttgacctcctctggctgattcaggaccggcccgaccgagacaatgacctacggttggcccagcacattacctatgtgcaccagcacagccggcagcccccctcccagtttgaacctctggacatgaagctcatgaggcgttacatagccatgtgccgcgagaagcagcccatggtgccagagtctctggctgactacatcacagcagcatacgtggagatgaggcgagaggcttgggctagtaaggatgccacctatacttctgcccggaccctgctggctatcctgcgcctttccactgctctggcacgtctgagaatggtggatgtggtggagaaagaagatgtgaatgaagccatcaggctaatggagatgtcaaaggactctcttctaggagacaaggggcagacagctaggactcagagaccagcagatgtgatatttgccaccgtccgtgaactggtctcagggggccgaagtgtccggttctctgaggcagagcagcgctgtgtatctcgtggcttcacacccgcccagttccaggcggctctggatgaatatgaggagctcaatgtctggcaggtcaatgcttcccggacacggatcacttttgtctgattccagcctgcttgcaaccctggggtcctcttgttccctgctggcctgccccttgggaaggggcagtgatgcctttgaggggaaggaggagcccctctttctcccatgctgcacttactccttttgctaataaaagtgtttgtagattgtcaaaaaaaaaaaaaaaaaaa

SEQ ID NO: 13 - DNA, 2447 bp, Homo sapiens
ccttggagccggatccggccccggaaacccgacctgcagacgcggtacctctactgcgtagaggccgtagctggcggaaggagagaggcggccgtcctgtcaacaggccgggggaagccgtgctttcgcggctgcccggtgcgacactttctccggacccagcatgtaggtgccgggcgactgccatgaactccggagccatgaggatccacagtaaaggacatttccagggtggaatccaagtcaaaaatgaaaaaaacagaccatctctgaaatctctgaaaactgataacaggccagaaaaatccaaatgtaagccactttggggaaaagtattttaccttgacttaccttctgtcaccatatctgaaaaacttcaaaaggacattaaggatctgggagggcgagttgaagaatttctcagcaaagatatcagttatcttatttcaaataagaaggaagctaaatttgcacaaaccttgggtcgaatttctcctgtaccaagtccagaatctgcatatactgcagaaaccacttcacctcatcccagccatgatggaagttcatttaagtcaccagacacagtgtgtttaagcagaggaaaattattagttgaaaaagctatcaaggaccatgattttattccttcaaatagtatattatcaaatgccttgtcatggggagtaaaaattcttcatattgatgacattagatactacattgaacaaaagaaaaaagagttgtatttactcaagaaatcaagtacttcagtaagagatgggggcaaaagagttggtagtggtgcacaaaaaacaagaacaggaagactcaaaaagccttttgtaaaggtggaagatatgagccaactttataggccattttatcttcagctgaccaatatgccttttataaattattctattcagaagccctgcagtccatttgatgtagacaagccatctagtatgcaaaagcaaactcaggttaaactaagaatccaaacagatggcgataagtatggtggaacctcaattcaactccagttgaaagagaagaagaaaaaaggatattgtgaatgttgcttgcagaaatatgaagatctagaaactcaccttctaagtgagcaacacagaaactttgcacagagtaaccagtatcaagttgttgatgatattgtatctaagttagtttttgactttgtggaatatgaaaaggacacacctaaaaagaaaagaataaaatacagtgttggatccctttctcctgtttctgcaagtgtcctgaaaaagactgaacaaaaggaaaaagtggaattgcaacatatttctcagaaagattgccaggaagatgatacaacagtgaaggagcagaatttcctgtataaagagacccaggaaactgaaaaaaagctcctgtttatttcagagcccatcccccacccttcaaatgaattgagagggcttaatgagaaaatgagtaataaatgttccatgttaagtacagctgaagatgacataagacagaattttacacagctacctctacataaaaacaaacaggaatgcattcttgacatttccgaacacacattaagtgaaaatgacttagaagaactaagggtagatcactataaatgtaacatacaggcatctgtacatgtttctgatttcagtacagataatagtggatctcaaccaaaacagaagtcagatactgtgctttttccagcaaaggatctcaaggaaaaggaccttcattcaatatttactcatgattctggtctgataacaataaacagttcacaagagcacctaactgttcaggcaaaggctccattccatactcctcctgaggaacccaatgaatgtgacttcaagaatatggatagtttaccttctggtaaaatacatcgaaaagtgaaaataatattaggacgaaatagaaaagaaaatctggaaccaaatgctgaatttgataaaagaactgaatttattacacaagaagaaaacagaatttgtagttcaccggtacagtctttactagacttgtttcagactagtgaagagaaatcagaatttttgggtttcacaagctacacagaaaagagtggtatatgcaatgttttagatatttgggaagaggaaaattcagataatctgttaacagcgtttttctcgtccccttcaacttctacatttactggcttttagaatttaaaaaatgcatacttttcagaagtgataaggatcatattcttgaaatttttataaatatgtatggaaattcttaggatttttttaccagctttgtttacagacccaaatgtaaatattaaaaataaatatttgcaattttctacagaattgaatacctgttaaagaaaaattacagaataaacttgtgactggtcttgttttacattaaaaaaaaaaaaaaaaaaaaaaaaa

SEQ ID NO: 14 - DNA, 2265 bp, Homo sapiens
gtggagtttgaattgggtggcggttgactgtagagccgctctctctcactggcacagcgaggttttgctcagcccttgtctcgggaccgcaggtacgtgcctggcgacttcttcgggtggtccccgtccgccctcctcgtccctacccagtttcttgcttccctgccccatctccgccgctccccgcagcctccgccgagcgccatggctcctaggaagggcagtagtcgggtggccaagaccaactccttacggaggcggaagctcgcctcctttctgaaagacttcgaccgtgaagtggaaatacgaatcaagcaaattgagtcagacaggcagaacctcctcaaggaggtggataacctctacaacatcgagatcctgcggctccccaaggctctgcgcgagatgaactggcttgactacttcgcccttggaggaaacaaacaggccctggaagaggcggcaacagctgacctggatatcaccgaaataaacaaactaacagcagaagctattcagacacccctgaaatctgccaaaacacgaaaggtaatacaggtagatgaaatgatagtggaagaggaagaagaagaagaaaatgaacgtaagaatcttcaaactgcaagagtcaaaaggtgtcctccatccaagaagagaactcagtccatacaaggaaaaggaaaagggaaaaggtcaagccgtgctaacactgttaccccagccgtgggccgattggaggtgtccatggtcaaaccaactccaggcctgacacccaggtttgactcaagggtcttcaagacccctggcctgcgtactccagcagcaggagagcggatttacaacatctcagggaatggcagccctcttgctgacagcaaagagatcttcctcactgtgccagtgggcggcggagagagcctgcgattattggccagtgacttgcagaggcacagtattgcccagctggatccagaggccttgggaaacattaagaagctctccaaccgtctcgcccaaatctgcagcagcatacggacccacaaatgagacaccaaagttgacaggatggacttttaatgggcacttctgggaccctgaagagacttcttcccttcaggcttattgtttgagtgtgaagttccagagcaaggagccatgttcctctaagggaattcaggaattcagacgtgctagtcccacaccagttaggtagagctgtctgttcaccctcccatcccagctgatcccagtcactgcttgctggggccatgccatggaagcttcccatcagtctcccagctgaatcctccctgctctctgagctgctgccttttgcctcctgcaactcaacatcctcttcaccctgccctgcctgcagttgagggggcgaagaagaaccctgtgttctcaggaagactgcctccaccaccgctacccagagaacctctgcatctggcatttctgctctctatgcttgagaccgggaggtttaggctcagataagtgagctctgggccatgagagggtaggtccagaaggtggggggaactgtacagatcagcagagcaggacagttggcagcagtgacctcagtagggaacatgtccgtctaccctctcgcactcatgacacctccccctaccagccctcctcttcctcctcctcctcctcctgtgggaggtggtcagtgggacttagggatctttcacctgctgtgcccagtagttctgaagtctgcttgtggagcagtgttttatgtttatccctgtttactgaagaccaaatactggtttggagacaacttccatgtcttgctcttctacctccctagttagtggaaatttggataagggaactgtagggcccagattctggaggttttatgtcattggccacagaataactgtctctaagctatccatggtccagtggtccctgccaagtctgtagacttcagagagcacttctctcttatggggttcatgggaacaggggcgggtgtgacttgcttggtggcctcattccatgtgtgcctgtgcctggggcatggactttgttaagcagagtcagcagtgaggtcctcattctccagccagcctctctgccctggagaatcatgtgctatgttctaagaatttgagaactagagtcctcatccccaggcttgaaggcacatggctttctcatgtagggctctctgtggtatttgttattattttgcaacaagaccattttagtaaaacaaaaaaaaaaaaaaaaaaaaaa

SEQ ID NO: 15 - DNA, 2459 bp, Homo sapiens
cttccctgtggtttcccgaggcctccttgcttcccgctctccgaggagcctttcatccgaaggcgggacgatgccggataatcggcagccgaggaaccggcagccgaggatccgctccgggaacgagcctcgttccgcgtccgccatggaacctgatggtcgcggtgcctgggcccacagtcgcgccgcgctcgaccgcctggagaagctgctgcgctgctcgcgttgtactaacattctgagagagcctgtgtgtttaggaggatgtgagcacatcttctgtagtaattgtgtaagtgactgcattggaactggatgtccagtgtgttacaccccggcctggatacaagacttgaagataaatagacaactggacagcatgattcaactttgtagtaagcttcgaaatttgctacatgacaatgagctgtcagatttgaaagaagataaacctaggaaaagtttgtttaatgatgcaggaaacaagaagaattcaattaaaatgtggtttagccctcgaagtaagaaagtcagatatgttgtgagtaaagcttcagtgcaaacccagcctgcaataaaaaaagatgcaagtgctcagcaagactcatatgaatttgtttccccaagtcctcctgcagatgtttctgagagggctaaaaaggcttctgcaagatctggaaaaaagcaaaaaaagaaaactttagctgaaatcaaccaaaaatggaatttagaggcagaaaaagaagatggtgaatttgactccaaagaggaatctaagcaaaagctggtatccttctgtagccaaccatctgttatctccagtcctcagataaatggtgaaatagacttactagcaagtggctccttgacagaatctgaatgttttggaagtttaactgaagtctctttaccattggctgagcaaatagagtctccagacactaagagcaggaatgaagtagtgactcctgagaaggtctgcaaaaattatcttacatctaagaaatctttgccattagaaaataatggaaaacgtggccatcacaatagactttccagtcccatttctaagagatgtagaaccagcattctgagcaccagtggagattttgttaagcaaacggtgccctcagaaaatataccattgcctgaatgttcttcaccaccttcatgcaaacgtaaagttggtggtacatcagggagcaaaaacagtaacatgtccgatgaattcattagtctttcaccaggtacaccaccttctacattaagtagttcaagttacaggcgagtgatgtctagtccctcagcaatgaagctgttgcccaatatggctgtgaaaagaaatcatagaggagagactttgctccatattgcttctattaagggcgacataccttctgttgaataccttttacaaaatggaagtgatccaaatgttaaagaccatgctggatggacaccattgcatgaagcttgcaatcatgggcacctgaaggtagtggaattattgctccagcataaggcattggtgaacaccaccgggtatcaaaatgactcaccacttcacgatgcagccaagaatgggcacatggatatagtcaagctgttactttcctatggagcctccagaaatgctgttaatatatttggtctgcggcctgtcgattatacagatgatgaaagtatgaaatcgctattgctgctaccagagaagaatgaatcatcctcagctagccactgctcagtaatgaacactgggcagcgtagggatggacctcttgtacttataggcagtgggctgtcttcagaacaacagaaaatgctcagtgagcttgcagtaattcttaaggctaaaaaatatactgagtttgacagtacagtaactcatgttgttgttcctggtgatgcagttcaaagtaccttgaagtgtatgcttgggattctcaatggatgctggattctaaaatttgaatgggtaaaagcatgtctacgaagaaaagtatgtgaacaggaagaaaagtatgaaattcctgaaggtccacgcagaagcaggctcaacagagaacagctgttgccaaagctgtttgatggatgctacttctatttgtggggaaccttcaaacaccatccaaaggacaaccttattaagctcgtcactgcaggtgggggccagatcctcagtagaaagcccaagccagacagtgacgtgactcagaccatcaatacagtcgcataccatgcgagacccgattctgatcagcgcttctgcacacagtatatcatctatgaagatttgtgtaattatcacccagagagggttcggcagggcaaagtctggaaggctccttcgagctggtttatagactgtgtgatgtcctttgagttgcttcctcttgacagctgaatattataccagatgaacatttcaaattgaatttgcacggtttgtgagagcccag

SEQ ID NO: 16 - DNA, 4214 bp, Homo sapiens
ctgagctgggtgggggtgccccacgctgaaagagagtgatggagtgcccagtgatggaaactgactcactttttacctcaggaattaagagacatttgaaagacaaaagaatttcaaagactactaagttgaatgtttctcttgcttcaaaaataaaaacaaaaatactaaataattcttctattttcaaaatatctttaaagcacaacaacagggcattagctcaggctcttagtagagaaaaagagaattctcgaagaattacaactgaaaagatgctattgcaaaaagaagtagagaaactgaattttgagaacacatttcttcgcctaaagctaaataacttgaataagaagcttatagacatagaagctctcatgaacaataacttgataactgcaattgaaatgagcagtctttctgagttccatcagagttcctttctactgtcagctagcaagaagaaacgaattagtaaacagtgcaagttgatgcgtcttccatttgcaagggttccattaacttcaaatgatgatgaagatgaagataaagagaaaatgcagtgtgacaacaatattaaatcaaagacattacctgatattccctcttcaggatcaacaacacaacctttatcaactcaggataattcggaagtgttatttcttaaagaaaataatcaaaatgtatatggtttagatgattcagaacatatttcttctatagttgatgtacctcccagagaaagccattcccactcagaccaaagttctaagacttctctaatgagtgagatgagaaacgcccagtctattggccgcagatgggagaaaccatctcctagtaatgtgactgaaaggaagaagcgtgggtcatcttgggaatcaaataatctttctgcagacactccctgtgcaacagttttagataaacaacacatttcaagtccagaattaaattgcaataatgagataaatggtcatactaatgaaacaaatactgaaatgcaaagaaataaacaggatcttcctggcttatcttctgagtctgccagagaacctaatgcagagtgcatgaatcaaattgaggataatgatgactttcaattgcagaaaactgtgtatgatgctgacatggatttaactgctagtgaagtcagcaaaattgtcacagtctcaacaggcattaaaaagaaaagtaataaaaaaacaaatgaacatggaatgaaaactttcagaaaagtgaaagattccagctctgaaaaaaagagagaaagatcaaagagacagtttaaaaatagttcagatgtcgatattggggaaaagattgaaaacaggacagaaagatctgatgtcctggatggcaaaaggggtgcagaagatcccggttttattttcaataatgaacagctggctcagatgaatgaacagctggctcaggtgaatgaactaaagaaaatgacccttcaaactggctttgaacaaggtgacagagaaaatgtactgtgtaataaaaaggagaaaagaataacaaatgagcaagaggaaacatactctttatcccaaagttcaggtaaatttcaccaggagagtaaatttgataagggtcagaattccctaacttgtaataaaagtaaagcttctagacagacatttgtgattcacaaattagaaaaagataacttactcccaaaccaaaaggataaagtaaccatttatgaaaacctagacgtcacaaatgaatttcacacagccaatctttccaccaaagataatggaaatttatgtgattatgggacccacaatatattggatttgaaaaagtatgtcactgatattcaaccctcagagcaaaatgaatcaaacattaataagcttagaaagaaagtaaaccggaagacagaaataatttctggaatgaaccacatgtatgaggataatgataaagatgtggtgcatggcctaaaaaaaggtaatttttttttcaaaacccaagaggataaagaacctatctctgaaaacatagaagtttccaaagagcttcaaatcccagctctttctactagagataatgaaaatcaatgtgactataggacccagaatgtgttgggtttgcaaaagcagatcaccaatatgtaccccgttcagcaaaatgaatcaaaagttaataagaagcttaggcagaaagtaaatcggaagacagaaataatttctgaagtgaatcatttagataatgacaaaagtatagaatacacagttaaaagtcactcactctttttaacgcaaaaagataaggaaatcatccctggaaacctagaagacccaagtgagtttgaaacacctgctctttctaccaaagatagtggaaacctgtatgattctgagattcaaaatgttttgggggtgaaacatggccatgatatgcaacctgcttgtcaaaatgattcaaaaataggtaagaagcctagactaaatgtatgtcaaaagtcagaaataattcctgaaaccaaccaaatatatgagaatgataacaaaggtgtacatgacctagaaaaagataacttcttctctctaaccccaaaggataaagaaacaatttctgaaaatctacaagtcacaaatgaatttcaaacagttgatcttctcatcaaagataatggaaatttatgtgattatgacacccagaatatattggagttgaaaaagtatgttactgataggaaatctgctgagcaaaatgaatcaaaaataaataagctcaggaataaagtgaattggaagacagaaataatttctgaaatgaaccagatatatgaggataatgataaagatgcacatgtccaagaaagctatacaaaagatcttgattttaaagtaaataaatctaaacaaaaacttgaatgccaagacattatcaataaacactatatggaagtcaacagtaatgaaaaggaaagttgtgatcaaattttagattcctacaaagtagttaaaaaacgtaagaaagaatcatcatgcaaggcaaagaacattttgacaaaagctaagaacaaacttgcttcacagttaacagaatcttcacagacatctatctccttagaatctgatttaaaacatattactagtgaagcagattctgatccaggaaacccagttgaactatgtaagactcagaagcaaagcactaccactttgaataaaaaagatctcccttttgtggaagaaataaaagaaggagagtgtcaggttaaaaaggtaaataaaatgacatctaagtcaaagaaaaggaagacctccatagatccttctccagagagccatgaagtaatggaaagaatacttgacagcgttcagggaaagtctactgtatctgaacaagctgataaggaaaacaatttggagaatgagaaaatggtcaaaaataagccagacttttacacaaaggcatttagatctttgtctgagatacattcacctaacatacaagattcttcctttgacagtgttcgtgaaggtttagtacctttgagcgtttcttctggtaaaaatgtgataataaaagaaaattttgccttggagtgctccccagcctttcaagtaagtgatgatgagcatgagaagatgaacaagatgaaatttaaagtcaaccggagaacccaaaaatcaggaataggtgatagaccattacaggacttgtcaaataccagttttgtttcaaataacactgctgaatctgaaaataagtcagaagatctatcttcagaacggacaagcagaagaagaaggtgtactcctttctattttaaagagccaagcctcagagacaagatgagaagatgaagtgaatttatggattctggtttttctgaattttcaaagcataaggaatcaaaacagaaatatagtatcaagaagatgaaatgcttaatgaaaaggttttttttttgtttctttggcctttcatggagtgttgatttgtccattcttaatgtttattaataggtatatgtgcataaaatagctattttgtaacattaaaccttttgagtcattttggtcatcatataacttaccttcctgtttatttaagcttctttttacctagtagcctttaaccaaacaataaccttttaaccaaataaaatgtgttaataaataaaaaaaaaaaaaaaaaaaaaaaaaaaaaaaaaaaaaaaaaaaaaaaaaaaaaaaaaaaaa

SEQ ID NO: 17 - DNA, 1697 bp, Homo sapiens
gaggcgtaagccaggcgtgttaaagccggtcggaactgctccggagggcacgggctccgtaggcaccaactgcaaggacccctccccctgcgggcgctcccatggcacagttcgcgttcgagagtgacctgcactcgctgcttcagctggatgcacccatccccaatgcaccccctgcgcgctggcagcgcaaagccaaggaagccgcaggcccggccccctcacccatgcgggccgccaaccgatcccacagcgccggcaggactccgggccgaactcctggcaaatccagttccaaggttcagaccactcctagcaaacctggcggtgaccgctatatcccccatcgcagtgctgcccagatggaggtggccagcttcctcctgagcaaggagaaccagcctgaaaacagccagacgcccaccaagaaggaacatcagaaagcctgggctttgaacctgaacggttttgatgtagaggaagccaagatccttcggctcagtggaaaaccacaaaatgcgccagagggttatcagaacagactgaaagtactctacagccaaaaggccactcctggctccagccggaagacctgccgttacattccttccctgccagaccgtatcctggatgcgcctgaaatccgaaatgactattacctgaaccttgtggattggagttctgggaatgtactggccgtggcactggacaacagtgtgtacctgtggagtgcaagctctggtgacatcctgcagcttttgcaaatggagcagcctggggaatatatatcctctgtggcctggatcaaagagggcaactacttggctgtgggcaccagcagtgctgaggtgcagctatgggatgtgcagcagcagaaacggcttcgaaatatgaccagtcactctgcccgagtgggctccctaagctggaacagctatatcctgtccagtggttcacgttctggccacatccaccaccatgatgttcgggtagcagaacaccatgtggccacactgagtggccacagccaggaagtgtgtgggctgcgctgggccccagatggacgacatttggccagtggtggtaatgataacttggtcaatgtgtggcctagtgctcctggagagggtggctgggttcctctgcagacattcacccagcatcaaggggctgtcaaggccgtagcatggtgtccctggcagtccaatgtcctggcaacaggagggggcaccagtgatcgacacattcgcatctggaatgtgtgctctggggcctgtctgagtgccgtggatgcccattcccaggtgtgctccatcctctggtctccccattacaaggagctcatctcaggccatggctttgcacagaaccagctagttatttggaagtacccaaccatggccaaggtggctgaactcaaaggtcacacatcccgggtcctgagtctgaccatgagcccagatggggccacagtggcatccgcagcagcagatgagaccctgaggctatggcgctgttttgagttggaccctgcgcggcggcgggagcgggagaaggccagtgcagccaaaagcagcctcatccaccaaggcatccgctgaagaccaacccatcacctcagttgttttttatttttctaataaagtcatgtctcccttcatgttttttttttaaaaaaaaaaaaaaaaaaaaaaaaa

SEQ ID NO: 18 - DNA, 1420 bp, Homo sapiens
gaagcaaggaggcggcggcggccgcagcgagtggcgagtagtggaaacgttgcttctgaggggagtccaagatgaccggttctaacgagttcaagctgaaccagccacccgaggatggcatctcctccgtgaagttcagccccaacacctcccagttcctgcttgtctcctcctgggacacgtccgtgcgtctctacgatgtgccggccaactccatgcggctcaagtaccagcacaccggcgccgtcctggactgcgccttctacgatccaacgcatgcctggagtggaggactagatcatcaattgaaaatgcatgatttgaacactgatcaagaaaatcttgttgggacccatgatgcccctatcagatgtgttgaatactgtccagaagtgaatgtgatggtcactggaagttgggatcagacagttaaactgtgggatcccagaactccttgtaatgctgggaccttctctcagcctgaaaaggtatataccctctcagtgtctggagaccggctgattgtgggaacagcaggccgcagagtgttggtgtgggacttacggaacatgggttacgtgcagcagcgcagggagtccagcctgaaataccagactcgctgcatacgagcgtttccaaacaagcagggttatgtattaagctctattgaaggccgagtggcagttgagtatttggacccaagccctgaggtacagaagaagaagtatgccttcaaatgtcacagactaaaagaaaataatattgagcagatttacccagtcaatgccatttcttttcacaatatccacaatacatttgccacaggtggttctgatggctttgtaaatatttgggatccatttaacaaaaagcgactgtgccaattccatcggtaccccacgagcatcgcatcacttgccttcagtaatgatgggactacgcttgcaatagcgtcatcatatatgtatgaaatggatgacacagaacatcctgaagatggtatcttcattcgccaagtgacagatgcagaaacaaaacccaagtcaccatgtacttgacaagatttcatttacttaagtgccatgttgatgataataaaacaattcgtactccccaatggtggatttattactattaaagaaaccagggaaaatattaattttaatattataacaacctgaaaataatggaaaagaggtttttgaatttttttttttaaataaacaccttcttaagtgcatgagatggtttgatggtttgctgcattaaaggtatttgggcaaacaaaattggagggcaagtgactgcagttttgagaatcagttttgaccttgatgattttttgtttccactgtggaaataaatgtttgtaaataagtgtaataaaaatccctttgcattcaaaaaaaaaaaaaaaaaa

SEQ ID NO: 19 - DNA, 3588 bp, Homo sapiens
gcggcgaccgtgaggccgagccgggagcgggcgtcttgccgaggcccgggcgggcggggagcaacggctacagacgccgcggggccaggtcgttgagggtcggcggcgggcgaggagcgcagggcgctcgggccgggggccgccggcgccatgggcaaccgcgggatggaagagctgatcccgctggtcaacaaactgcaggacgccttcagctccatcggccagagctgccacctggacctgccgcagatcgctgtagtgggcggccagagcgccggcaagagctcggtgctggagaacttcgtgggccgggacttccttccccgcggttcaggaatcgtcacccggcggcctctcattctgcagctcatcttctcaaaaacagaacatgccgagtttttgcactgcaagtccaaaaagtttacagactttgatgaagtccggcaggagattgaagcagagaccgacagggtcacggggaccaacaaaggcatctccccagtgcccatcaaccttcgagtctactcgccacacgtgttgaacttgaccctcatcgacctcccgggtatcaccaaggtgcctgtgggcgaccagcctccagacatcgagtaccagatcaaggacatgatcctgcagttcatcagccgggagagcagcctcattctggctgtcacgcccgccaacatggacctggccaactccgacgccctcaagctggccaaggaagtcgatccccaaggcctacggaccatcggtgtcatcaccaagcttgacctgatggacgagggcaccgacgccagggacgtcttggagaacaagttgctcccgttgagaagaggctacattggcgtggtgaaccgcagccagaaggatattgagggcaagaaggacatccgtgcagcactggcagctgagaggaagttcttcctctcccacccggcctaccggcacatggccgaccgcatgggcacgccacatctgcagaagacgctgaatcagcaactgaccaaccacatccgggagtcgctgccggccctacgtagcaaactacagagccagctgctgtccctggagaaggaggtggaggagtacaagaactttcggcccgacgaccccacccgcaaaaccaaagccctgctgcagatggtccagcagtttggggtggattttgagaagaggatcgagggctcaggagatcaggtggacactctggagctctccgggggcgcccgaatcaatcgcatcttccacgagcggttcccatttgagctggtgaagatggagtttgacgagaaggacttacgacgggagatcagctatgccattaagaacatccatggagtcaggaccgggcttttcaccccggacttggcattcgaggccattgtgaaaaagcaggtcgtcaagctgaaagagccctgtctgaaatgtgtcgacctggttatccaggagctaatcaatacagttaggcagtgtaccagtaagctcagttcctacccccggttgcgagaggagacagagcgaatcgtcaccacttacatccgggaacgggaggggagaacgaaggaccagattcttctgctgatcgacattgagcagtcctacatcaacacgaaccatgaggacttcatcgggtttgccaatgcccagcagaggagcacgcagctgaacaagaagagagccatccccaatcaggtgatccgcaggggctggctgaccatcaacaacatcagcctgatgaaaggcggctccaaggagtactggtttgtgctgactgccgagtcactgtcctggtacaaggatgaggaggagaaagagaagaagtacatgctgcctctggacaacctcaagatccgtgatgtggagaagggcttcatgtccaacaagcacgtcttcgccatcttcaacacggagcagagaaacgtctacaaggacctgcggcagatcgagctggcctgtgactcccaggaagacgtggacagctggaaggcctcgttcctccgagctggcgtctaccccgagaaggaccaggcagaaaacgaggatggggcccaggagaacaccttctccatggacccccaactggagcggcaggtggagaccattcgcaacctggtggactcatacgtggccatcatcaacaagtccatccgcgacctcatgccaaagaccatcatgcacctcatgatcaacaatacgaaggccttcatccaccacgagctgctggcctacctatactcctcggcagaccagagcagcctcatggaggagtcggctgaccaggcacagcggcgggacgacatgctgcgcatgtaccatgccctcaaggaggcgctcaacatcatcggtgacatcagcaccagcactgtgtccacgcctgtacccccgcctgtcgatgacacctggctccagagcgccagcagccacagccccactccacagcgccgaccggtgtccagcatacacccccctggccggcccccagcagtgaggggccccactccagggccccccctgattcctgttcccgtgggggcagcagcctccttctcggcgcccccaatcccatcccggcctggaccccagagcgtgtttgccaacagtgacctcttcccagccccgcctcagatcccatctcggccagttcggatccccccagggattcccccaggagtgcccagcagaagaccccctgctgcgcccagccggcccaccattatccgcccagccgagccatccctgctcgactaggcctcgaggggggcgtgctctcgggggggcctcacgcacccgcggcgcaggagcttcagtggtctggggccctccgccgcccctatgctgggaccaggctcccagtgggcagccctggcctcttccttaacgctggccccggtccagggccggcccctgtgcctggctggacaccgcactgcgcaaaggggccctggagctccaggcagggggcgctggggtgttgcactttgggggatggagtctcagggtggcagaggggggaccagaacccttgacaccatcctgaatgaggggtccagcctgggggggactctaccaaggtcttcttgggctgggaaagcccatgtagggcaggccttctataagtgcgggcaccaagggcgcctacatccccaggccttgctggggtgcaggggtatatcaacttcccattagcaggagctccccagcggcaagcctggcccagtgggctcggtagtgcccagctggcaggcctgaggtgtacatagtccttcccggccatattaaccacacagcctgagcctggcccagcctcggctgccagaggtgcctttgctaggcccggagccgttggcccgggccggccttgccctattcctctcctcctcctcctcctgggtcccccagggtggctgggcttgggctatgtgggtggtggtggcggggggtcttgggggcctctcagctcccgcccatgcctccctgatgggtgggcccagggcggcctctctctgaggagacctcacccactcctcgctcagtttgaccactgtaagtgcctgcactctgtattctattaaaaaaaaaaaaaaa

SEQ ID NO: 20 - DNA, 5101 bp, Homo sapiens
agcgcagccattggtccggctactctgtctctttttcaaattgaggcgccgagtcgttgcttagtttctggggattcgggcggagacgagattagtgatttggcggctccgactggcgcgggacaaacgccacggccagagtaccgggtagagagcggggacgccgacctgcgtgcgtcggtcctccaggccacgccagcgcccgagagggaccagggagactccggcccctgtcggccgccaagcccctccgcccctcacagcgcccaggtccgcggccgggccttgattttttggcggggaccgtcatggcgtcgcagccaaattcgtctgcgaagaagaaagaggagaaggggaagaacatccaggtggtggtgagatgcagaccatttaatttggcagagcggaaagctagcgcccattcaatagtagaatgtgatcctgtacgaaaagaagttagtgtacgaactggaggattggctgacaagagctcaaggaaaacatacacttttgatatggtgtttggagcatctactaaacagattgatgtttaccgaagtgttgtttgtccaattctggatgaagttattatgggctataattgcactatctttgcgtatggccaaactggcactggaaaaacttttacaatggaaggtgaaaggtcacctaatgaagagtatacctgggaagaggatcccttggctggtataattccacgtacccttcatcaaatttttgagaaacttactgataatggtactgaattttcagtcaaagtgtctctgttggagatctataatgaagagctttttgatcttcttaatccatcatctgatgtttctgagagactacagatgtttgatgatccccgtaacaagagaggagtgataattaaaggtttagaagaaattacagtacacaacaaggatgaagtctatcaaattttagaaaagggggcagcaaaaaggacaactgcagctactctgatgaatgcatactctagtcgttcccactcagttttctctgttacaatacatatgaaagaaactacgattgatggagaagagcttgttaaaatcggaaagttgaacttggttgatcttgcaggaagtgaaaacattggccgttctggagctgttgataagagagctcgggaagctggaaatataaatcaatccctgttgactttgggaagggtcattactgcccttgtagaaagaacacctcatgttccttatcgagaatctaaactaactagaatcctccaggattctcttggagggcgtacaagaacatctataattgcaacaatttctcctgcatctctcaatcttgaggaaactctgagtacattggaatatgctcatagagcaaagaacatattgaataagcctgaagtgaatcagaaactcaccaaaaaagctcttattaaggagtatacggaggagatagaacgtttaaaacgagatcttgctgcagcccgtgagaaaaatggagtgtatatttctgaagaaaattttagagtcatgagtggaaaattaactgttcaagaagagcagattgtagaattgattgaaaaaattggtgctgttgaggaggagctgaatagggttacagagttgtttatggataataaaaatgaacttgaccagtgtaaatctgacctgcaaaataaaacacaagaacttgaaaccactcaaaaacatttgcaagaaactaaattacaacttgttaaagaagaatatatcacatcagctttggaaagtactgaggagaaacttcatgatgctgccagcaagctgcttaacacagttgaagaaactacaaaagatgtatctggtctccattccaaactggatcgtaagaaggcagttgaccaacacaatgcagaagctcaggatatttttggcaaaaacctgaatagtctgtttaataatatggaagaattaattaaggatggcagctcaaagcaaaaggccatgctagaagtacataagaccttatttggtaatctgctgtcttccagtgtctctgcattagataccattactacagtagcacttggatctctcacatctattccagaaaatgtgtctactcatgtttctcagatttttaatatgatactaaaagaacaatcattagcagcagaaagtaaaactgtactacaggaattgattaatgtactcaagactgatcttctaagttcactggaaatgattttatccccaactgtggtgtctatactgaaaatcaatagtcaactaaagcatattttcaagacttcattgacagtggccgataagatagaagatcaaaaaaaggaactagatggctttctcagtatactgtgtaacaatctacatgaactacaagaaaataccatttgttccttggttgagtcacaaaagcaatgtggaaacctaactgaagacctgaagacaataaagcagacccattcccaggaactttgcaagttaatgaatctttggacagagagattctgtgctttggaggaaaagtgtgaaaatatacagaaaccacttagtagtgtccaggaaaatatacagcagaaatctaaggatatagtcaacaaaatgacttttcacagtcaaaaattttgtgctgattctgatggcttctcacaggaactcagaaattttaaccaagaaggtacaaaattggttgaagaatctgtgaaacactctgataaactcaatggcaacctggaaaaaatatctcaagagactgaacagagatgtgaatctctgaacacaagaacagtttatttttctgaacagtgggtatcttccttaaatgaaagggaacaggaacttcacaacttattggaggttgtaagccaatgttgtgaggcttcaagttcagacatcactgagaaatcagatggacgtaaggcagctcatgagaaacagcataacatttttcttgatcagatgactattgatgaagataaattgatagcacaaaatctagaacttaatgaaaccataaaaattggtttgactaagcttaattgctttctggaacaggatctgaaactggatatcccaacaggtacgacaccacagaggaaaagttatttatacccatcaacactggtaagaactgaaccacgtgaacatctccttgatcagctgaaaaggaaacagcctgagctgttaatgatgctaaactgttcagaaaacaacaaagaagagacaattccggatgtggatgtagaagaggcagttctggggcagtatactgaagaacctctaagtcaagagccatctgtagatgctggtgtggattgttcatcaattggcggggttccatttttccagcataaaaaatcacatggaaaagacaaagaaaacagaggcattaacacactggagaggtctaaagtggaagaaactacagagcacttggttacaaagagcagattacctctgcgagcccagatcaacctttaattcacttgggggttggcaattttatttttaaagaaaacttaaaaataaaacctgaaaccccagaacttgagccttgtgtatagattttaaaagaatatatatatcagccgggcgcggtggctcatgcctgtaatcccagcactttgggaggctgaggcgggtggattgcttgagcccaggagtttgagaccagcctggccaacgtggcaaaacctcgtctctgttaaaaattagccgggcgtggtggcacactcctgtaatcccagctactggggaggctgaggcacgagaatcacttgaacccaggaagcggggttgcagtgagccaaaggtacaccactacactccagcctgggcaacagagcaagactcggtctcaaaaacaaaatttaaaaaagatataaggcagtactgtaaattcagttgaattttgatatctacccatttttctgtcatccctatagttcactttgtattaaattgggtttcatttgggatttgcaatgtaaatacgtatttctagttttcatataaagtagttcttttataacaaatgaaaagtatttttcttgtatattattaagtaatgaatatataagaactgtactcttctcagcttgagcttacataggtaaatatcaccaacatctgtccttagaaaggaccatctcatgttttttttcttgctatgacttgtgtattttcttgcatcctccctagacttccctatttcgctttctcctcggctcactttctccctttttatttttcaccaaaccatttgtagagctacaaaaggtatcctttcttattttcagtagtcagaattttatctagaaatcttttaacacctttttagtggttatttctaaaatcactgtcaacaataaatctaaccctagttgtatccctcctttcagtatttttcacttgttgccccaaatgtgaaagcatttcattcctttaagaggcctaactcattcaccctgacagagttcacaaaaagcccacttaagagtatacattgctattatgggagaccacccagacatctgactaatggctctgtgcccacactccaagacctgtgccttttagagaagctcacaatgatttaaggactgtttgaaacttccaattatgtctataatttatattcttttgtttacatgatgaaactttttgttgttgcttgtttgtatataatacaatgtgtacatgtatctttttctcgattcaaatcttaacccttaggactctggtatttttgatctggcaaccatatttctggaagttgagatgtttcagcttgaagaaccaaaacagaaggaatatgtacaaagaataaattttctgctcacgatgagtttagtgtgtaaagtttagagacatctgactttgatagctaaattaaaccaaaccctattgaagaattgaatatatgctacttcaagaaactaaattgatctcgtagaattatcttaataaaataatggctataatttctctgcaaaatcagatgtcagcataagcgatggataatacctaataaactgccctcagtaaatccatggttaataaatgtggtttctacattaaaaaaaaaaaaaaaaaaaaaaaaa

SEQ ID NO: 21 - DNA, 10661 bp, Homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaacaaaaaaaaaaagcaaaaacaaaacaaaaaataagccaaaaaaccttgctagtgttttttcctcaaaaataaataaataaataaataaatacgtacatacatacacacatacatacaaacatatagaaatccccaaagaggccaatagtgacgagaaggtgaaaattgcaggcccatggggagttactgattttttcatctcctccctccacgggagactttattttctgccaatggctattgccattagagggcagagtgaccccagagctgagttgggcaggggggtggacagagaggagaggacaaggagggcaatggagcatcagtacctgcccacagccttggtccctgggggctagactgctcaactgtggagcaattcattatactgaaaatgtgcttgttgttgaaaatttgtctgcatgttaatgcctcacccccaaacccttttctctctcactctctgcctccaacttcagattgactttcaatagtttttctaagacctttgaactgaatgttctcttcagccaaaacttggcgacttccacagaaaagtctgaccactgagaagaaggagagcagagatttaaccctttgtaaggccccatttggatccaggtctgctttctcatgtgtgagtcagggaggagctggagccagaggagaagaaaatgatagcttggctgttctcctgcttaggacactgactgaatagttaaactctcactgccactaccttttccccacctttaaaagacctgaatgaagttttctgccaaactccgtgaagccacaagcaccttatgtcctcccttcagtgttttgtgggcctgaatttcatcacactgcatttcagccatggtcatcaagcctgtttgcttcttttgggcatgttcacagattctctgttaagagcccccaccaccaagaaggttagcaggccaacagctctgacatctatctgtagatgccagtagtcacaaagatttcttaccaactctcagatcgctggagcccttagacaaactggaaagaaggcatcaaagggatcaggcaagctgggcgtcttgcccttgtcccccagagatgataccctcccagcaagtggagaagttctcacttccttctttagagcagctaaaggggctacccagatcagggttgaagagaaaactcaattaccagggtgggaagaatgaaggcactagaaccagaaaccctgcaaatgctcttcttgtcacccagcatatccacctgcagaagtcatgagaagagagaaggaacaaagaggagactctgactactgaattaaaatcttcagcggcaaagcctaaagccagatggacaccatctggtgagtttactcatcatcctcctctgctgctgattctgggctctgacattgcccatactcactcagattccccacctttgttgctgcctcttagtcagagggaggccaaaccattgagactttctacagaaccatggcttctttcggaaaggtctggttggtgtggctccaatactttgccacccatgaactcagggtgtgccctgggacactggttttatatagtcttttggcacacctgtgttctgttgacttcgttcttcaagcccaagtgcaagggaaaatgtccacctactttctcatcttggcctctgcctccttacttagctcttaatctcatctgttgaactcaagaaatcaagggccagtcatcaagctgcccattttaattgattcactctgtttgttgagaggatagtttctgagtgacatgatatgatccacaagggtttccttccctgatttctgcattgatattaatagccaaacgaacttcaaaacagctttaaataacaagggagaggggaacctaagatgagtaatatgccaatccaagactgctggagaaaactaaagctgacaggttccctttttggggtgggatagacatgttctggttttctttattattacacaatctggctcatgtacaggatcacttttagctgttttaaacagaaaaaaatatccaccactcttttcagttacactaggttacattttaataggtcctttacatctgttttggaatgattttcatcttttgtgatacacagattgaattatatcattttcatatctctccttgtaaatactagaagctctcctttacatttctctatcaaatttttcatctttatgggtttcccaattgtgactcttgtcttcatgaatatatgtttttcatttgcaaaagccaaaaatcagtgaaacagcagtgtaattaaaagcaacaactggattactccaaatttccaaatgacaaaactagggaaaaatagcctacacaagcctttaggcctactctttctgtgcttgggtttgagtgaacaaaggagattttagcttggctctgttctcccatggatgaaaggaggaggattttttttttcttttggccattgatgttctagccaatgtaattgacagaagtctcattttgcatgcgctctgctctacaaacagagttggtatggttggtatactgtactcacctgtgagggactggccactcagacccacttagctggtgagctagaagatgaggatcactcactggaaaagtcacaaggaccatctccaaacaagttggcagtgctcgatgtggacgaagagtgaggaagagaaaaagaaggagcaccagggagaaggctccgtctgtgctgggcagcagacagctgccaggatcacgaactctgtagtcaaagaaaagagtcgtgtggcagtttcagctctcgttcattgggcagctcgcctaggcccagcctctgagctgacatgggagttgttggattctttgtttcatagctttttctatgccataggcaatattgttgttcttggaaagtttattatttttttaactcccttactctgagaaagggatattttgaaggactgtcatatatctttgaaaaaagaaaatctgtaatacatatatttttatgtatgttcactggcactaaaaaatatagagagcttcattctgtcctttgggtagttgctgaggtaattgtccaggttgaaaaataatgtgctgatgctagagtccctctctgtccatactctacttctaaatacatataggcatacatagcaagttttatttgacttgtactttaagagaaaatatgtccaccatccacatgatgcacaaatgagctaacattgagcttcaagtagcttctaagtgtttgtttcattaggcacagcacagatgtggcctttccccccttctctcccttgatatctggcagggcataaaggcccaggccacttcctctgccccttcccagccctgcaccaaagctgcatttcaggagactctctccagacagcccagtaactacccgagcatggcccctgcatagccctggaaaaataagaggctgactgtctacgaattatcttgtgccagttgcccaggtgagagggcactgggccaagggagtggttttcatgtttgacccactacaaggggtcatgggaatcaggaatgccaaagcaccagatcaaatccaaaacttaaagtcaaaataagccattcagcatgttcagtttcttggaaaaggaagtttctacccctgatgcctttgtaggcagatctgttctcaccattaatctttttgaaaatcttttaaagcagtttttaaaaagagagatgaaagcatcacattatataaccaaagattacattgtacctgctaagataccaaaattcataagggcagggggggagcaagcattagtgcctctttgataagctgtccaaagacagactaaaggactctgctggtgactgacttataagagctttgtgggtttttttttccctaataatatacatgtttagaagaattgaaaataatttcgggaaaatgggattatgggtccttcactaagtgattttataagcagaactggctttccttttctctagtagttgctgagcaaattgttgaagctccatcattgcatggttggaaatggagctgttcttagccactgtgtttgctagtgcccatgttagcttatctgaagatgtgaaacccttgctgataagggagcatttaaagtactagattttgcactagagggacagcaggcagaaatccttatttctgcccactttggatggcacaaaaagttatctgcagttgaaggcagaaagttgaaatacattgtaaatgaatatttgtatccatgtttcaaaattgaaatatatatatatatatatatatatatatatatatatatatagtgtgtgtgtgtgttctgatagctttaactttctctgcatctttatatttggttccagatcacacctgatgccatgtacttgtgagagaggatgcagttttgttttggaagctctctcagaacaaacaagacacctggattgatcagttaactaaaagttttctcccctattgggtttgacccacaggtcctgtgaaggagcagagggataaaaagagtagaggacatgatacattgtactttactagttcaagacagatgaatgtggaaagcataaaaactcaatggaactgactgagatttaccacagggaaggcccaaacttggggccaaaagcctacccaagtgattgaccagtggccccctaatgggacctgagctgttggaagaagagaactgttccttggtcttcaccatccttgtgagagaagggcagtttcctgcattggaacctggagcaagcgctctatctttcacacaaattccctcacctgagattgaggtgctcttgttactgggtgtctgtgtgctgtaattctggttttggatatgttctgtaaagattttgacaaatgaaaatgtgtttttctctgttaaaacttgtcagagtactagaagttgtatctctgtaggtgcaggtccatttctgcccacaggtagggtgtttttctttgattaagagattgacacttctgttgcctaggacctcccaactcaaccatttctaggtgaaggcagaaaaatccacattagttactcctcttcagacatttcagctgagataacaaatcttttggaattttttcacccatagaaagagtggtagatatttgaatttagcaggtggagtttcatagtaaaaacagcttttgactcagctttgatttatcctcatttgatttggccagaaagtaggtaatatgcattgattggcttctgattccaattcagtatagcaaggtgctaggttttttcctttccccacctgtctcttagcctggggaattaaatgagaagccttagaatgggtggcccttgtgacctgaaacacttcccacataagctacttaacaagattgtcatggagctgcagattccattgcccaccaaagactagaacacacacatatccatacaccaaaggaaagacaattctgaaatgctgtttctctggtggttccctctctggctgctgcctcacagtatgggaacctgtactctgcagaggtgacaggccagatttgcattatctcacaaccttagcccttggtgctaactgtcctacagtgaagtgcctggggggttgtcctatcccataagccacttggatgctgacagcagccaccatcagaatgacccacgcaaaaaaaagaaaaaaaaaattaaaaagtcccctcacaacccagtgacacctttctgctttcctctagactggaacattgattagggagtgcctcagacatgacattcttgtgctgtccttggaattaatctggcagcaggagggagcagactatgtaaacagagataaaaattaattttcaatattgaaggaaaaaagaaataagaagagagagagaaagaaagcatcacacaaagattttcttaaaagaaacaattttgcttgaaatctctttagatggggctcatttctcacggtggcacttggcctccactgggcagcaggaccagctccaagcgctagtgttctgttctctttttgtaatcttggaatcttttgttgctctaaatacaattaaaaatggcagaaacttgtttgttggactacatgtgtgactttgggtctgtctctgcctctgctttcagaaatgtcatccattgtgtaaaatattggcttactggtctgccagctaaaacttggccacatcccctgttatggctgcaggatcgagttattgttaacaaagagacccaagaaaagctgctaatgtcctcttatcattgttgttaatttgttaaaacataaagaaatctaaaatttcaaaaaa